(19) United States
(12) Patent Application Publication
(10) Pub. No.: US 2026/0196323 A1
(43) Pub. Date: Jul. 9, 2026

(54) ADVERSE DRUG DETECTION USING ONTOLOGY-AUGMENTED LARGE LANGUAGE MODELS

(71) Applicant: Oracle International Corporation, Redwood Shores, CA (US)

(72) Inventors: Suman Roy, Bangalore (IN); Shirish Amit Bajpai, Bengaluru (IN); Srijon Sarkar, Bangalore (IN)

(73) Assignee: Oracle International Corporation, Redwood Shores, CA (US)

(21) Appl. No.: 19/009,253

(22) Filed: Jan. 3, 2025

(51) Int. Cl. G16H 20/10; G06F 18/2413; G06N 5/01; G16H 10/60

(52) U.S. Cl. CPC G16H 20/10; G06F 18/24147; G06N 5/01; G16H 10/60

(57) ABSTRACT

Described are techniques for adverse drug detection using ontology-augmented Large Language Models (LLM). In one aspect, a method is provided that includes accessing medical text associated with a patient. The medical text is annotated for drug and event entities to create labelled medical text, and spans in the labelled medical text are linked to concepts in an ontology. Possible paths in the ontology are identified based on the labeling and linking, each of the possible paths are ranked, and a top number of paths are identified based on the ranking. A verbalized form of each path of the top number of paths are then concatenated with associated portion(s) of the medical text to create ontology augmented text instances and a prompt having the ontology augmented text instance(s) are generated. Thereafter, one or more adverse drug reaction relation predictions are generated, by an LLM, for the patient based on the prompt.

### FIELD

The present disclosure relates generally to adverse drug detection, and more particularly, to techniques for adverse drug detection using ontology-augmented Large Language Models (LLMs).

### BACKGROUND

In a robust medication safety program, data is collected and reported, then analyzed to examine trends and outliers and implement changes that hopefully make systems better and improve safety. Adverse Drug Events (ADE) is the general term that encompasses all medication-related incidents (both Adverse Drug Reaction (ADR) and medication errors) and is defined as any untoward occurrence that may show up during treatment with a pharmaceutical product which does not necessarily have a causal relationship with this treatment. ADRs are a type of ADE and are defined as any any noxious change which is suspected to be due to a drug, occurs at doses normally used in a man, requires treatment or decrease in dose or indicates caution in future use of the same drug. Therefore, an ADR is an ADE with a causal link to a drug. Medication errors are another type of ADE and are preventable events that may cause inappropriate medication use or patient harm. These can occur at any stage in the medication use process, such as prescribing/ordering, communication, storage and labeling, compounding, dispensing/distribution, administration, and monitoring. In a medication error, at least one step in the process had an excursion from what should have happened.

Despite the importance of identifying ADRs, they are often underreported in Electronic Health Records (EHRs). This underreporting poses a significant health problem worldwide, as it limits the ability of healthcare providers and researchers to fully understand the scope and nature of ADRs. In the United States alone, over 2 million serious ADRs reportedly occur among hospitalized patients annually. These figures highlight the critical need for improved systems and methodologies to detect and monitor ADRs effectively.

One promising approach to mitigating the incidence of ADRs is the implementation of systems within hospitals and health centers to monitor ADEs regularly. Advances in computational techniques, particularly in Natural Language Processing (NLP), have led to the development of various methods for detecting ADRs in clinical texts. These techniques range from rule-based systems to classical machine learning approaches and more sophisticated deep learning techniques. Each of these methods offers unique advantages and challenges, but collectively, they represent significant progress in the field of ADR detection.

Large Language Models (LLMs) have recently brought a transformational change to the computational approach in NLP, offering new possibilities for ADR detection and monitoring. Models such as GPT-4 have demonstrated remarkable abilities across a wide range of tasks, including those specific to healthcare applications. These models can analyze and interpret vast amounts of clinical data with high accuracy, potentially identifying ADRs that might be missed by traditional methods. The integration of LLMs into ADR detection systems represents a promising avenue for enhancing drug safety and patient care through more reliable and comprehensive monitoring of drug reactions.

### SUMMARY

Described herein are embodiments (e.g., a method, a system, non-transitory computer-readable medium storing code or instructions executable by one or more processors) pertaining to techniques for adverse drug detection using ontology-augmented Large Language Models (LLMs).

In various embodiments, a computer-implemented method is provided for that comprises: accessing medical text associated with a patient; annotating spans in the medical text for drug and event entities to create labelled medical text; linking, using entity linking, the spans in the labelled medical text to concepts in an ontology, wherein the linking comprises: for a subject span in the labelled medical text with a mapped entity type of drug or event, link the subject span to a most similar subject concept in the ontology, and for an object span in the labelled medical text with a mapped entity type of drug or event, link the object span to a most similar object concept in the ontology; for each pair of linked subject and object spans, identifying possible paths in the ontology between the subject concept and the object concept, ranking each of the possible paths in the ontology, identifying a top number of paths from all the possible paths based on the ranking, and concatenating a verbalized form of each path of the top number of paths with the subject pan and the object span from the labelled medical text to create an ontology augmented text instance corresponding to the pair of linked subject and object spans; generating a prompt comprising the medical text and each ontology augmented text instance; generating, by a generative artificial intelligence model, one or more adverse drug reaction relation predictions for the patient based on the prompt; and providing the one or more adverse drug reaction relation predictions to a user.

In some embodiments, the ontology comprises concepts, relations, and one or more triplets having a subject concept, a relation, and an object concept, and wherein a path in the ontology is a sequence of concepts and relations identified therein by associated one or more triplets.

In some embodiments, the computer-implemented method further comprises: retrieving, based on a query of ontology data, an ontology associated with the medical text; verbalizing, using one or more templates, the one or more triplets in the ontology to represent each of the one or more triplets in form of a textual description; embedding the textual description for each of the one or more triplets; and storing the embedded textual description for each of the one or more triplets in a vector database, wherein the linking is performed between the spans in the labelled medical text to the concepts in the embedded textual description for each of the one or more triplets stored in the vector database.

In some embodiments, a determination of whether a subject span or an object span in the labelled medical text with the mapped entity type of drug or event is most similar to a corresponding subject concept or object concept in the ontology is performed using an approximate nearest neighbor (ANN) search, and wherein the ANN search performs a semantic similarity search in the vector database using the subject spans and the object spans as query data points.

In some embodiments, ranking each of the possible paths in the ontology comprises: verbalizing each of the identified possible paths in the ontology by concatenating the textual description for the one or more triplets associated with each of the identified possible paths; concatenating each pair of the linked subject and object spans with the associated verbalized form of each of the identified possible paths to generate a corresponding possible path input; determining, by a pre-trained cross-encoder, a scaler value for each possible path input; and ranking the possible path inputs based on the corresponding scalar value for each possible path input, wherein the top number of paths are identified from all the possible paths based on the ranking of the possible path inputs.

In some embodiments, the prompt further comprises an overview of the adverse drug reaction relation task and intermediate texts as part of in-context learning, and wherein the generative artificial intelligence model is a LLM fine-tuned for the adverse drug reaction relation task.

In some embodiments, the one or more adverse drug reaction relation predictions are provided to a user as part of plug-in for an application associated with a cloud service.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein.

Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is an illustration of an ADR-LLM is a framework in accordance with various embodiments.

FIG. 2 shows a portion of a EHR with spans indicating DRUG and ADE and thus showing presence of ADR or not in accordance with various embodiments.

FIG. 3 shows a block diagram illustrating an AI platform for training and deploying models in accordance with various embodiments.

FIG. 4 shows an Electronic Medical Record (EMR)/EHR analyzer tool implementation in accordance with various embodiments.

FIG. 5 shows an overview of ADR prediction (RE prediction) framework with KG-augmented LLM inference in accordance with various embodiments.

FIG. 6 shows a snippet of ADE ontology in accordance with various embodiments.

FIG. 7 shows construction of a base ontology from a collection of clinical narratives on ADE in accordance with various embodiments.

FIG. 8 shows a schematic diagram of RAG-assisted ADE prediction on LLM in accordance with various embodiments.

FIG. 9 shows a bar graph illustrating effectiveness of a supplemental prompt in ADR prediction in accordance with various embodiments.

FIG. 10 depicts a flowchart illustrating a process for adverse drug detection using ontology-augmented LLMs in accordance with various embodiments.

FIG. 11 is a block diagram illustrating one pattern for implementing a cloud infrastructure as a service system, according to at least one embodiment.

FIG. 12 is a block diagram illustrating another pattern for implementing a cloud infrastructure as a service system, according to at least one embodiment.

FIG. 13 is a block diagram illustrating another pattern for implementing a cloud infrastructure as a service system, according to at least one embodiment.

FIG. 14 is a block diagram illustrating another pattern for implementing a cloud infrastructure as a service system, according to at least one embodiment.

FIG. 15 is a block diagram illustrating an example computer system, according to at least one embodiment.

### DETAILED DESCRIPTION

In the following description, for the purposes of explanation, specific details are set forth in order to provide a thorough understanding of certain inventive embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

### Summary

Described herein are techniques for how LLMs can be efficiently used to predict ADRs. These techniques include augmenting knowledge from relevant drug ontology (in the form of a knowledge graph) directly in the input of LLM for the task of ADR detection. More specifically, the techniques retrieve concepts in a relevant ontology corresponding to an input pair of entities based on semantic similarity between the two, create triplets on the recovered concepts, and prepend the sequence of retrieved ontologies to the input in the form of a prompt, which is then fed to LLMs to predict ADRs. The effect of using knowledge from ontologies on ADR prediction was evaluated using a public biomedical dataset, ADE. The ontology augmented LLM inference on ADRs shows an improvement performance over LLM-based ADR prediction lacking ontology-augmentation and other baseline approaches.

### Challenges Solved

An Adverse Event (AE) is any undesirable experience associated with the use of a medical product in a patient. As explained above, an ADR is an unintended and harmful reaction which is suspected to be due to the routine use of drugs under normal conditions. It should be understood that an ADR is a type of ADE, which is a type of AE, and the terms ADE and ADR are preferably used hereafter to facilitate an explanation of the techniques described herein. However, it should be understood further that the techniques described herein are applicable to any type of AE.

A significant public health problem arises due to ADR throughout the world. In the United States, it is reported that over 2 million serious ADRs occur among hospitalized patients which results in over 10,000 deaths every year. It is desired to detect potential ADRs on drug candidates in the early stage of the drug development pipeline as this can improve drug safety, reduce risks for the patients and save money for the pharmaceutical companies. The incidents of ADRs can be reduced if hospitals and health centers use systems to monitor ADE occurrences regularly.

Researchers have used a variety of computational techniques around Natural Language Processing (NLP) for predicting ADRs for clinical texts e.g., using rule-based systems, classical machine learning-based techniques, and of late deep learning techniques. The task of ADR may be performed by leveraging models of distributional semantics—i.e., unsupervised methods that exploit co-occurrence information to model, typically in vector space, the meaning of words, which leads to the improvement of the predictive performance. However, more recently context information is better modeled by transformer models through attention and the availability of labeled data somehow lessens the importance of this effort. Researchers have used supervised NLP methods to detect ADE mentions in clinical notes to improve medication safety in hospitalized patients (see, Murphy R. M., Klopotowska J. E., de Keizer N. F., Jager K. J., Leopold J. H., Dongelmans D. A., Abu-Hanna A., Schut M. C. Adverse drug event detection using natural language processing: A scoping review of supervised learning methods. PLOS One. 2023 Jan. 3; 18(1)). However, this study has led to the conclusion that more work is needed for data preparation and deployment stages of NLP methods. Further, researchers have used deep learning models which can simultaneously predict ADRs and identify the molecular substructures associated with those ADRs without defining the substructures a-priori (see, Sanjoy Dey, Heng Luo, Achille Fokoue, Jianying Hu, Ping Zhang: Predicting adverse drug reactions through interpretable deep learning framework. BMC Bioinform. 19-S(21): 476:1-476:13 (2018)). However, this work requires significant effort in the creation of fingerprint feature representations.

In order to address these challenges, the use of LLMs has been proposed for ADR detection to mitigate the drawbacks of the prior approaches. Large language models (LLMs), such as GPT-4, have performed remarkably on a wide range of tasks, including health applications as these models have brought disruptive changes in the computational NLP approaches. As the ADR problem can be cast as an information retrieval problem in NLP, LLMs can be expected to provide a better result for ADR prediction. Nonetheless, LLMs models alone may struggle to understand the relationships between drugs and their adverse effects.

More specifically, an end-to-end ADR prediction involves two separate NLP subtasks—(1) identifying AE mentions and drug name mentions typically using named entity recognition (NER), where a drug causation is not yet assigned, and (2) assigning causation to drugs typically through relation extraction (RE) which predicts relations between AEs and corresponding drugs. For purposes of the following problem definition, the presence of mentions of both AEs and drug names is assumed in the input text, and the task of supervised ADR prediction is the primary focus. These notions may be formalized briefly as follows.

Assume the input to the framework is a document represented as a sequence of words: D={w₁, w₂, . . . , wₙ}. Construct S={s₁, s₂, . . . , s_{N}}, the set of all possible within-sentence word sequence spans (up to a reasonable length) in the documents. Let E denote a set of pre-defined entity types. In this case E={AE,Drug}∪{ϵ}, where AE is an entity type denoting adverse event, Drug is an entity type denoting Medicine Name; ϵ stands for null entity. Assume some of the spans in the text will be mapped to entity types AE or Drug; rest of the spans will be assigned null entity type. Hence the input text will be associated with a set of augmented entities Yₑ={(sᵢ, e): sᵢ∈S, e∈E}. This will be also called the set of labelled entities.

Take an example text: “Patient is on Amlong 10 mg. Complaining swelling of feet and hair loss”. In this text the span ‘Amlong’ is mapped to entity type Drug, ‘swelling of feet’ and ‘hair loss’ mapped to AE. All other spans are mapped to null entity type.

ADR prediction may be defined as a relation prediction problem. Let R denote a set of pre-defined relation types. In this case R will be comprised of two relations, ADR and No-ADR. Let D be the input sentence. The goal is, for every pair (sᵢ, eₖ)∈Yₑ, and (sⱼ, eₗ)∈Yₑ, appearing in D predict a relation type yᵣ(sᵢ, sⱼ)∈R, i.e., R(sᵢ, sⱼ). The output is given as

$${Y}_{R}^{S} = \{ ( {s}_{i} , r , {s}_{j} ) \text{:} {s}_{i} , {s}_{j} ∈ S , r ∈ R \}$$

etc.

For the input text above, ADR prediction will result in the following relations, ADR (Amlong, swelling of feet) and No-ADR (Amlong, hair loss).

Thus, ADR relation extraction aims to establish causal links between the extracted AE entities and drugs in the context or otherwise. This step is needed to understand the relationships between drugs and their adverse effects, which can enable enabling more informed decisions related to drug safety and usage.

### Overview of Embodiments and Solution for Challenges

Embodiments described herein for an ADR-LLM framework, addresses the aforementioned challenges and others by enhancing the LLM's ability to understand the relationships between drugs and their adverse effects. More specifically, the ADR-LLM framework addresses the aforementioned challenges and others by augmenting knowledge from relevant Drug Ontology (in the form of Knowledge Graph) directly in the input of an LLM for ADR detection. The ontology augmented LLM inference on ADRs shows an improvement performance over LLM-based ADR prediction without ontology and other baseline approaches.

The ADR-LLM framework utilizes fine-tuning of LLMs with specific instructions and contextual prompts to identify relationships between drugs and ADEs in clinical texts. The framework models the ADR prediction task by annotating input text instances with spans representing drugs and ADEs and determining whether these spans are linked by an ADR or No-ADR relation. The process includes generating detailed prompts that integrate supplementary information for in-context learning, enabling the model to consider broader contextual factors such as comorbidities and concomitant medications during ADR detection.

Additionally, the ADR-LLM framework incorporates a knowledge graph (KG) in the form of a biomedical ontology to enhance the accuracy of ADR predictions. This ontology is comprised of controlled vocabularies that describe the semantics of data in a machine-readable format, which is then transformed into textual sequences to be used by the LLM. The KG-augmented approach involves retrieving relevant paths within the ontology, which are then fed into the LLM for inference. Experimental results demonstrate that integrating ontology knowledge into the LLM significantly improves the precision, recall, and F1-score of ADR predictions compared to models that do not utilize ontology augmentation. This framework can be adapted for both zero-shot and fine-tuned ADR prediction scenarios, offering a robust solution for detecting adverse drug reactions in medical texts.

In various embodiments, a computer implemented method is provided for that comprises: accessing medical text associated with a patient; annotating spans in the medical text for drug and event entities to create labelled medical text; linking, using entity linking, the spans in the labelled medical text to concepts in an ontology, where the linking comprises: for a subject span in the labelled medical text with a mapped entity type of drug or event, link the subject span to a most similar subject concept in the ontology, and for an object span in the labelled medical text with a mapped entity type of drug or event, link the object span to a most similar object concept in the ontology; for each pair of linked subject and object spans, identifying possible paths in the ontology between the subject concept and the object concept, ranking each of the possible paths in the ontology, identifying a top number of paths from all the possible paths based on the ranking, and concatenating a verbalized form of each path of the top number of paths with the subject pan and the object span from the labelled medical text to create an ontology augmented text instance corresponding to the pair of linked subject and object spans; generating a prompt comprising the medical text and each ontology augmented text instance; generating, by a generative artificial intelligence model, one or more adverse drug reaction relation predictions for the patient based on the prompt; and providing the one or more adverse drug reaction relation predictions to a user.

As used herein, the terms “about,” “similarly,” “substantially,” and “approximately” are defined as being largely but not necessarily wholly what is specified (and include wholly what is specified) as understood by one of ordinary skill in the art. In any disclosed embodiment, the term “about,” “similarly,” “substantially,” or “approximately” may be substituted with “within [a percentage] of” what is specified, where the percentage includes 0.1 percent, 1 percent, 5 percent, and 10 percent, etc. Moreover, the term terms “about,” “similarly,” “substantially,” and “approximately” are used to provide flexibility to a numerical range endpoint by providing that a given value may be slightly above or slightly below the endpoint without affecting the desired result.

As used herein, when an action is “based on” something, this means the action can be based at least in part on at least a part of the something.

### Overview of the ADR-LLM Framework

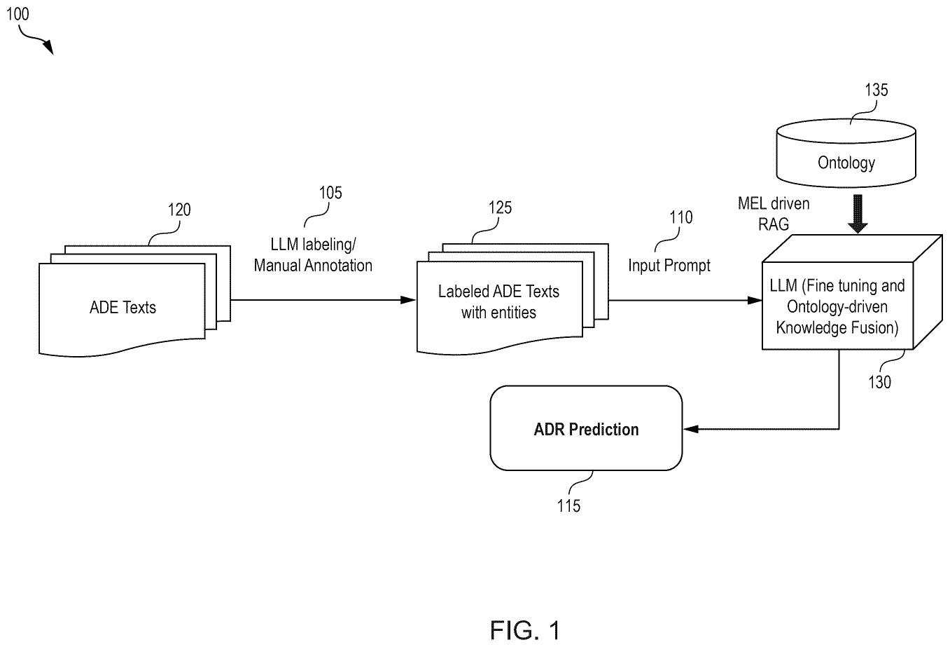

The ADR-LLM is a framework for modeling an ADR prediction task using fine tuning and ontology driven knowledge fusion. As shown in FIG. 1, the workflow 100 for the ADR-LLM framework 100 comprises the following steps: input annotation 105, prompt construction 110, and completion 115.

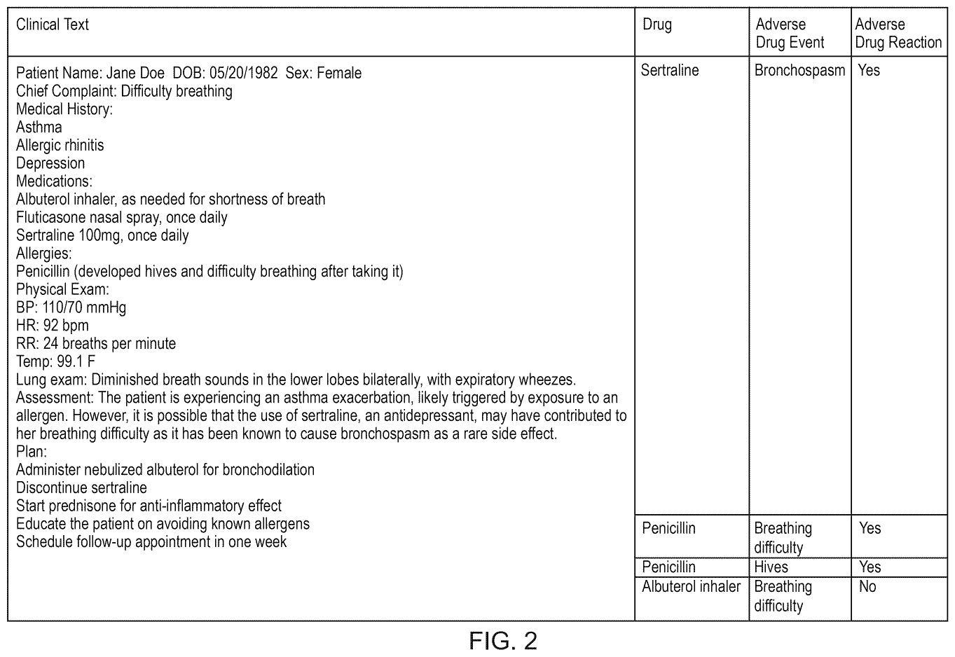

For the input annotation 105, each input medical text instance 120 (e.g., medical record or portion thereof) is annotated to identify spans representing Drugs and ADEs. A “span” refers to a sequence of tokens (e.g., a contiguous sequence of words) in a text or document. A token is a unit of text that has been extracted from the input text during the tokenization process, which involves breaking down a piece of text into individual words, punctuation marks, or other meaningful elements. Thus, a span in natural language processing is essentially a sequence of these tokens, e.g., a contiguous sequence of words. Spans can represent entities, phrases, or any other segment of text that is relevant to the analysis or task at hand. During training certain spans are linked by either an ADR relation or a No-ADR relation. An input medical text instance 120 may be medical text associated with a patient's health such as an electronic health record (EHR), clinical trial data, research data, the like, or any combination thereof (FIG. 2 shows an example of medical text in a portion of patient's EHR). Annotation of the input medical text instances 120 creates labelled ADE texts with entities 125.

For example, given an input medical text instance: adr_text=“Patient is on Amlong 10 mg. Complaining of swelling of feet and hair loss”, the annotation (Drug, ADE; Relation: ADR, No-ADR) may be as follows:
Span of Drug: “Amlong”
Span of ADE: “swelling of feet”
Span of ADE: “hair loss”

A prompt is then constructed for the labelled ADE texts with entities 125 using prompt construction 110. The prompt includes an overview of the ADR detection task, the identified spans, and the corresponding relations. Each prompt may also integrate supplementary information for in-context learning, which aids a model 130 in considering broader contextual factors such as comorbidities and concomitant medications.

Continuing with the example above, given an input medical text instance: adr_text=“Patient is on Amlong 10 mg. Complaining of swelling of feet and hair loss” and the labelled ADE texts with entities 125, a prompt may be constructed as follows:

TABLE 1
Example of Prompts for ADR Prediction

| | |
|---|---|
| Prompt | You are ChatPharmacovigilance, an expert ADR detection BOT. Your task is to |
| | identify Adverse drug reaction from adr text. |
| | We consider two entity types: <Drug> and <ADE>. The entity type Drug |
| | corresponds to Medicine name. |
| | The entity type Adverse Drug Event (ADE) is a drug event or a drug reaction |
| | which is an unintended reaction or |
| | event suspected to be due to the routine use of drugs under normal conditions. |
| | 1. You need to assign a causation of drug to ADE by predicting a relation |
| | between Drug and ADE. For that we introduce two types of relations between |
| | entity types Drug and ADE, they are <ADR> (Adverse Durg Reaction) and <No- |
| | ADR>. |
| | 2. Consider the spans in the input text that are mapped to entity type Drug (or |
| | Medicine Name) or DE. |
| | 3. If you find the relation ADR between the span mapped to Drug and the span |
| | mapped to DE then respond with relation ADR, |
| | else respond with relation No-ADR. |
| | Format your response with JSON objects with “Relation”, “Span of Drug” and |
| | “Span of ADE’ as the keys. |
| | If the information is not available use “unknown” as the value. |
| | Make your response as short as possible.\ |
| | adr text: ″′{adr_text}′″ |
| | adr_text = <Patient is on Amlong 10 mg. Complaining of swelling of feet and |
| | hair loss> |
| | { |
| | “Span of Drug”: “Amlong” |
| | “Span of ADE”: “swelling of feet” |
| | “Span of ADE”: “hair loss” |
| | } |
| Supplementary | Please take into account the following facts for your task: |
| information | 1. Consider contextual information of the entire text to decide. It should not be |
| | based just on the similarity |
| | between ADE and Drug |
| | 2. Consider effect of comorbidity and concomitant medications, the reaction |
| | could be as well due to them and |
| | not actually is the ADE due to a drug. |
| | 3 If a Drug does not cure the condition or behave in intended way, respond with |
| | ADR as well. |
| | 4 If the withdrawal of a drug results in deterioration of the patient then respond |
| | with No-ADR. |
| Completion | Your task is to identify Adverse drug reaction from the adr text. |
| | Format your response with JSON objects with “Relation”, “Span of Drug” and |
| | “Span of ADE’ as the keys. |
| | If the information is not available use “unknown” as the value. |
| | Make your response as short as possible.\ |
| | adr text: ″′{ adr_text}′″ |
| | adr_text = <Patient is on Amlong 10 mg. Complaining of swelling of feet and |
| | hair loss> |
| | { |
| | “Span of Drug”: “Amlong” |
| | “Span of ADE”: “swelling of feet” |
| | “Span of ADE”: “hair loss” |
| | } |
| | Output ADR: |
| | “Relation”: “ADR” |
| | “Span of Drug”: “Amlong” |
| | “Span of Adv DE”: “swelling of feet” |
| | “Relation”: “No-ADR” |
| | “Span of Drug”: “Amlong” |
| | “Span of Adv DE”: “hair loss” |

The model 130 is then tasked with determining a relation between the identified spans as a completion 115 (i.e., output), considering the prompt and ontology 135 created from a collection of clinical narratives on the ADE. For an input medical text instance, useful knowledge (context for the prompt) is infused into the model 130 from the ontology 135 (i.e., KG) by retrieving useful facts using medical entity linking (MEL) in terms of appropriate paths in the ontology 135 (in the framework of Retrieval-Augmented Generation (RAG)). The ontology 135 provides structural knowledge, domain specific knowledge, and evolving knowledge to help the model 130 with accuracy, decisiveness, and interpretability of the input medical text instance 120.

Continuing with the example above, given an input medical text instance: adr_text=“Patient is on Amlong 10 mg. Complaining of swelling of feet and hair loss”, the labelled ADE texts with entities, the constructed prompt, and the ontology, one or more ADR predictions may be made as follows:

Output ADR prediction by a model may be as follows:
“Relation”: “ADR”/*ADR(Amlong, swelling of feet)*/causal link
   “Span of Drug”: “Amlong”
   “Span of ADE”: “swelling of feet”
“Relation”: “No-ADR”/*No-ADR(Amlong, hair loss)*/no causal link
   “Span of Drug”: “Amlong”
   “Span of ADE”: “hair loss”

### Input Annotation

Annotating text data involves the process of labeling or tagging text with relevant information that can guide the model's learning process. This typically includes providing clear and structured data pairs, where each input (prompt) is matched with the correct or desired output (completion). The annotations may include various forms of metadata, such as parts of speech, named entities, or specific instructions on how the text should be interpreted or processed. For example, for each input medical text, each of the one or more spans and the entities (ADE and Drug) may be specified with annotations to generate labeled text. The annotations may also be generated to identify whether these spans are connected by the relation ADR or No-ADR (i.e., ground truth label). By annotating text data such as the input medical text, the model is provided with a rich and informative dataset that helps it understand the relationships and patterns necessary to generate accurate and contextually appropriate responses. This structured approach ensures that the model can produce high-quality completions that align closely with the intended outcomes (i.e., ADR prediction).

### Prompt Construction

Constructing a prompt involves designing the input to include not only the text that the model needs to process but also clear instructions and in some instances illustrative examples. The construction process can be a manual, automated (e.g., scripts and/or logic for building templates and slot-filling), or semi-automated process (e.g., scripts and/or logic for building templates and slot-filling with a human in the loop to facilitate and validate prompt construction). The structured prompt guides the model on how to approach the task, providing context and clarity. For instance, a prompt can start with an instruction explaining what is expected, followed by an example input and its corresponding correct output. This combination of text, instructions, and examples helps the model understand the desired behavior and output format, thereby improving its performance and accuracy during the fine-tuning process. For example, a prompt is constructed for each labelled ADE text with entities (i.e., training example), which is fed to the model. Each prompt is constructed to comprise the following components: the input text (e.g., clinical or medical text), a succinct overview of the ADR task description, and one or more ADR relations (e.g., two relations) that are to be considered. The model is explicitly asked in the prompt instruction to output a relation, which is at least one of the one or more ADR relations.

In some instances, the prompt is constructed to further comprise supplementary information. The supplementary information may include intermediate texts as part of in-context learning for each of the labelled ADE text with entities. The intermediate texts are a prompt engineering technique that aims to improve language models' performance on tasks requiring logic, calculation and decision-making similar to a chain of thought prompting technique, which enables complex reasoning capabilities through intermediate reasoning steps. The supplementary information can be thought of as a kind of tacit knowledge to be extracted by the model for a dataset. For example, the following intermediate texts may be included as part of the in-context learning.
Consider contextual information of the entire text to make a decision. It should not be based just on closeness of ADR and Drug
Consider effect of comorbidity and concomitant medications, the reaction could be as a result of them and not actually ADR due to a Drug
If a Drug does not cure the condition or behave in intended way, consider it as ADR as well. All inputs from now on will be Input text to be analyzed, provide response in correct output format
If the withdrawal of a drug results in deterioration of the patient, then respond with ‘No-ADR’.

### Completion

A completion as used herein refers to the text output generated by the model as a response to a user's input or prompt. Essentially, the completion is the completed text that the LLM produces based on the provided context (e.g., ontology) and information within the prompt. For example, for a given prompt and ontology, the completion is the model's attempt to provide an answer (i.e., one or more ADR relations) based on its training and/or fine-tuning. These completions are essentially predictions made by the model about what comes next in a sequence of text, based on patterns it has learned during its training phase.

### AI Platform

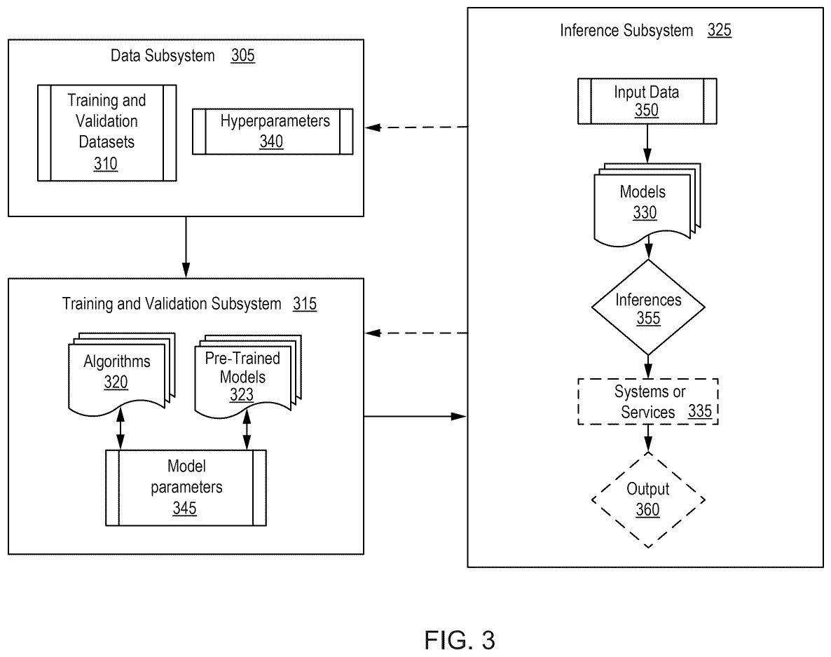

FIG. 3 shows a block diagram of a machine learning pipeline 300 comprising several subsystems that work together to train, validate, and implement one or more machine learning models in accordance with various embodiments. The machine learning pipeline 300 may be executed as part of the ADR-LLM framework 100 described in FIG. 1 to fine-tune one or more machine learning models (e.g., LLMs) with instructions (i.e., instruction tuning). More specifically, one challenge of using LLMs for a prediction task, is the mismatch between the training objective and users' objective. While base LLMs are typically trained on minimizing the contextual word prediction error on a large corpus, in a prediction task the user desires the model to be able to follow their instructions helpfully and safely. This challenge is addressed herein by using instruction tuning, which serves as an effective technique to enhance the capabilities and controllability of the LLMs.

The machine learning pipeline 300 comprises a data subsystem 305 for collecting, generating, preprocessing, and labeling of training and validation datasets 310, training and validation subsystem 315 that facilitates the training and validation of one or more machine learning algorithms 320 or one or more pre-trained machine learning models 323, and inference subsystem 325 for deploying and implementing one or more trained machine learning models 330 independently or in combination with one or more other systems or services 335 for downstream processes.

As used herein, machine learning algorithms (also described herein as simply algorithm or algorithms) are procedures that are run on datasets (e.g., training and validation datasets) and perform pattern recognition on datasets, learn from the datasets, and/or are fit on the datasets. Examples of machine learning algorithms include linear and logistic regression, decision trees, artificial neural networks, k-means, transformer architectures with attention mechanisms, and k-nearest neighbor. In contrast, machine learning models (also described herein as simply model or models) are the output of the machine learning algorithms and are comprised of model data and a prediction algorithm. In other words, the machine learning model is the program that is saved after running a machine learning algorithm on training data and represents the rules, numbers, and any other algorithm-specific data structures required to make inferences. For example, a linear regression algorithm may result in a model comprised of a vector of coefficients with specific values, and a transformer architecture with attention mechanisms may result in a LLM that utilizes self-attention mechanisms, allowing the model to weigh the importance of different words in a sentence when making predictions.

In the specific context of this disclosure, the machine learning model(s) may be LLMs. An LLM is an advanced type of machine learning model designed to understand and generate human-like text based on vast amounts of training data. These models, such as GPT-4, are trained on diverse datasets that include books, articles, and websites, enabling them to learn patterns, context, and nuances of language. LLMs can perform a wide range of tasks, including answering questions, translating languages, summarizing text, and even generating creative content, by predicting the next word or sequence of words in a given context. Their ability to generate coherent and contextually appropriate responses makes them valuable tools in various applications, from customer service to content creation and beyond such as in this disclosure predicting one or more ADR relations within medical text.

In certain instances, some of the models described herein are pre-trained LLM models that are being fine-tuned on training data. Pre-trained LLMs are advanced neural network models that have been trained on vast amounts of text data to understand and generate human-like language. These models, such as GPT-4, Lama, and T5, undergo a pre-training phase where they learn the statistical properties and patterns of language from large-scale corpora, which can include books, articles, websites, and other textual data. During this phase, the models are not trained for any specific task but rather to predict the next word in a sentence or to understand sentence structure, semantics, and context, resulting in a rich general understanding of language. Once pre-trained, these models can be fine-tuned for specific downstream tasks such as text classification, question answering, translation, summarization, and as described herein prediction of ADR relations. Fine-tuning involves further training the pre-trained model on a smaller, task-specific dataset, allowing it to adapt its generalized language understanding to the particular nuances and requirements of the task at hand. This approach leverages the extensive linguistic knowledge captured during the pre-training phase, significantly enhancing the model's performance and efficiency on specialized applications with relatively limited data compared to training from scratch.

### Data Subsystem

Data subsystem 305 is used to collect, generate, preprocess, and label data to be used to train and validate one or more machine learning algorithms 320 or one or more pre-trained machine learning models 323. The data collection can include exploring various data sources such as public datasets, private data collections, or real-time data streams, depending on a project's needs. In some instances, a data source is a public or online repository of information or examples pertinent to a general or target domain space (e.g., medical text from electronic health records (EHRs)). Many domains have publicly available datasets provided by governments, universities, or organizations. For example, many government and private entities offer datasets on healthcare, environmental data, and more through various portals. For proprietary needs, data might be available through partnerships or purchases from private companies that specialize in data aggregation. In other instances, a data source is a private repository of information or examples pertinent to a general or target domain space. For example, a data source can be the storage device that stores digital EHRs or other sources of digital medical text accessed by the ADR-LLM framework 100 described in FIG. 1. Once a data source is identified, data subsystem 305 can be used to collect data through appropriate methods such as downloading from online repositories, web scraping, using APIs for real-time data, creating datasets through surveys and requests for access, or by running programs or scripts. The acquired raw data may be further preprocessed to generate the training and validation datasets 310.

In some instances, raw data may be generated as opposed to being collected or acquired. Data generating may comprise data synthesis and/or data augmentation. Different data synthesis and/or data augmentation techniques may be implemented by the data subsystem 305 to generate data to be used for the training and validation subsystem 315. Data synthesizing involves creating entirely new data points from scratch. This technique may be used when real data is insufficient, too sensitive to use, or when the cost and logistical barriers to obtaining more real data are too high. The synthesized data should be realistic enough to effectively train a machine learning model, but distinct enough to comply with regulations (e.g., copyright and data privacy), if necessary. Techniques such as Generative Adversarial Networks (GANs) or Variational Autoencoders (VAEs) may be used to generate new data examples. These models learn the distribution of real data and attempt to produce new data examples that are statistically similar but not identical. Data augmentation, on the other hand, refers to techniques used to artificially expand the size of a dataset by creating modified versions of existing data examples. The primary goal of data augmentation is to increase variation in the data in order to make the model more robust to variations it might encounter in the real world, thereby improving its ability to generalize from the training data to unseen data. This is especially common in image and speech recognition tasks but is applicable to other data types as well. For images, data augmentation may include rotations, flipping, scaling, or altering the lighting conditions. For text, data augmentation may include synonyms replacement, back translation, or sentence shuffling. For audio, data augmentation may include changes made to pitch, speed, or background noise.

Preprocessing may be implemented by the data subsystem 305, serving as a bridge between raw data acquisition and effective model training. The primary objective of preprocessing is to transform raw data into a format that is more suitable and efficient for analysis, ensuring that the data fed into machine learning algorithms or pretrained models is clean, consistent, and relevant. This step can be useful because raw data often comes with a variety of issues such as missing values, noise, irrelevant information, and inconsistencies that can significantly hinder the performance of a model. By standardizing and cleaning the data beforehand, preprocessing helps in enhancing the accuracy and efficiency of the subsequent analysis, making the data more representative of the underlying problem the model aims to solve.

Other raw data preprocessing techniques that may be utilized include data cleaning, normalization, feature extraction, dimensionality reduction, and the like. Data cleaning may involve removing duplicates, filling in missing values, or filtering out outliers to improve data quality. Normalization involves scaling numeric values to a common scale without distorting differences in the ranges of values, which helps prevent biases in the model due to the inherent scale of features. Feature extraction involves transforming the input data into a set of useable features, possibly reducing the dimensionality of the data in the process. For instance, in image analysis, feature reduction techniques such as Principal Component Analysis (PCA), Linear Discriminant Analysis (LDA), t-Distributed Stochastic Neighbor Embedding (t-SNE), autoencoders, and feature selection can be used for simplifying images, improving model performance, and gaining insights into the underlying structure of the images. These techniques not only help in reducing the computational load on the model but also in mitigating issues like overfitting by simplifying the data without losing critical information.

In the instance that machine learning pipeline 300 is used for supervised or semi-supervised learning of machine learning models, labeling techniques can be implemented as part of the data collection. The quality and accuracy of data labeling directly influence the model's performance, as labels serve as the definitive guide that the model uses to learn the relationships between the input features and the desired output. Particularly in complex domains such as image analysis, natural language processing, or medical diagnosis, precise and consistent labeling is important because it provides the ground truth or target outcomes against which the model's predictions are compared and adjusted during training. Effective labeling ensures that the model is trained on correct and clear examples, thus enhancing its ability to generalize from the training data to real-world scenarios. In some instances, the annotation labels and ground truth values (labels) are appended or annotated within the raw data as described with respect to FIG. 1. For example, when the raw data includes medical text, the labels may include the one or more spans, corresponding entities (ADE and Drug), and whether these spans are connected by the relation ADR or No-ADR (i.e., ground truth label).

Labeling techniques can vary significantly depending on the type of data and the specific requirements of the project. Manual labeling, where human annotators label the data, is one method that can be used. This approach may be useful when a detailed understanding and judgment are required, such as in labeling medical images or categorizing text data where context and subtlety are important. However, manual labeling can be time-consuming and prone to inconsistency, especially with a large number of annotators. To mitigate this, semi-automated labeling tools may be used as part of data subsystem 305 to pre-label data using algorithms, which human annotators may then review and correct as needed. Another approach is active learning, a technique where the model being developed is used to label new data iteratively. The model suggests labels for new data points, and human annotators may review and adjust certain predictions such as the most uncertain predictions. This technique optimizes the labeling effort by focusing human resources on a subset of the data, e.g., the most ambiguous cases, improving efficiency and label quality through continuous refinement.

Once collected, generated, preprocessed, and/or labeled, the data may then be split into the training and validation datasets 310. The training and validation datasets 310 may comprise the raw data and/or the preprocessed data. The training and validation datasets 310 are typically split into at least three subsets of data: training, validation, and testing. The training set is used to fit the model, where the machine learning model learns to make inferences based on the training data. The validation set, on the other hand, is utilized to tune hyperparameters and prevent overfitting by providing a sandbox for model selection. Finally, the test set serves as a new and unseen dataset for the model, used to simulate real-world application and evaluate the final model's performance. The process of splitting ensures that the model can perform well not just on the data it was trained on, but also on new, unseen data, thereby validating and testing its ability to generalize.

Various techniques can be employed to split the data effectively, with each method aiming to maintain a good representation of the overall dataset in each subset. A simple random split (e.g., a 70/20/10%, 80/10/10%, or 60/25/15%) is the most straightforward approach, where examples from the data are randomly assigned to each of the three sets. In some instances, the splitting is performed such that 70% of the training and validation datasets 310 are for training, 10% are for validation, and 20% are for testing. However, more sophisticated methods may be necessary to preserve the underlying distribution of data. For instance, stratified sampling may be used to ensure that each split reflects the overall distribution of a specific variable, particularly useful in cases where certain categories or outcomes are underrepresented. Another technique, k-fold cross-validation, involves rotating the validation set across different subsets of the data, maximizing the use of available data for training while still holding out portions for validation. These methods help in achieving more robust and reliable model evaluation and are useful in the development of predictive models that perform consistently across varied datasets.

Data subsystem 305 is also used for collecting, generating, setting, or implementing model hyperparameters 340 for the training and validation subsystem 315. The hyperparameters control the overall behavior of the models. Unlike model parameters 345 that are learned automatically during training, hyperparameters 340 are set before training begins and have a significant impact on the performance of the model. For example, in a neural network such as that of an LLM, hyperparameters include the learning rate, number of layers, number of neurons/nodes per layer, activation functions, convolution kernel width, the number of kernels for a model, among others. These settings can determine how quickly a model learns, its capacity to generalize from training data to unseen data, and its overall complexity. Correctly setting hyperparameters is important because inappropriate values can lead to models that underfit or overfit the data. Underfitting occurs when a model is too simple to learn the underlying pattern of the data, and overfitting happens when a model is too complex, learning the noise in the training data as if it were signal.

### Training, Validation, and Testing

The training and validation subsystem 315 is comprised of a combination of specialized hardware and software to efficiently handle the computational demands required for training, validating, and testing a machine learning model. On the hardware side, high-performance GPUs (Graphics Processing Units) may be used for their ability to perform parallel processing, drastically speeding up the training of complex models, especially deep learning networks. CPUs (Central Processing Units), while generally slower for this task, may also be used for less complex model training or when parallel processing is less critical. TPUs (Tensor Processing Units), designed specifically for tensor calculations, provide another level of optimization for machine learning tasks. On the software side, a variety of frameworks and libraries are utilized, including TensorFlow, PyTorch, Keras, and scikit-learn. These tools offer comprehensive libraries and functions that facilitate the design, training, validation, and testing of a wide range of machine learning models across different computing platforms, whether local machines, cloud-based systems, or hybrid setups, enabling developers to focus more on model architecture and less on underlying computational details.

Training is the initial phase of developing machine learning models 330 where the model learns to make predictions or decisions based on data training data provided from the training and validation datasets 310. During this phase, the model iteratively adjusts its internal model parameters 345 to achieve a preset optimization condition. In a supervised machine learning training process, the preset optimization condition can be achieved by minimizing the difference between the model output (e.g., predictions, classifications, or decisions) and the ground truth labels in the training data. In some instances, the preset optimization condition can be achieved when the preset fixed number of iterations or epochs (full passes through the training dataset) is reached. In some instances, the preset optimization condition is achieved when the performance on the validation dataset stops improving or starts to degrade. In some instances, the preset optimization condition is achieved when a convergence criterion is met, such as when the change in the model parameters falls below a certain threshold between iterations. This process, known as fitting, is fundamental because it directly influences the accuracy and effectiveness of the model.

In an exemplary training phase performed by the training and validation subsystem 315, the training subset of data is input into the machine learning algorithms 320 or pre-trained models 323 to find a set of model parameters 345 (e.g., weights, coefficients, trees, feature importance, and/or biases) that minimizes or maximizes an objective function (e.g., a loss function, a cost function, a contrastive loss function, a cross-entropy loss function, an Out-of-Bag (OOB) score, etc.). To train the machine learning algorithms 320 or pre-trained models 323 to achieve accurate predictions, “errors” (e.g., a difference between a predicted label and the ground truth label) need to be minimized. In order to minimize the errors, the model parameters can be configured to be incrementally updated by minimizing the objective function over the training phase (“optimization”). Various different techniques may be used to perform the optimization. For example, to train machine learning algorithms or pre-trained models such as a neural network, optimization can be done using back propagation. The current error is typically propagated backwards to a previous layer, where it is used to modify the weights and bias in such a way that the error is minimized. The weights are modified using the optimization function. Other techniques such as random feedback, Direct Feedback Alignment (DFA), Indirect Feedback Alignment (IFA), Hebbian learning, and the like can also be used to update the model parameters 345 in a manner as to minimize or maximize an objective function. This cycle is repeated until a desired state (e.g., a predetermined minimum value of the objective function) is reached.

The training phase is driven by three primary components: the model architecture (which defines the structure of the algorithm(s) 320 or pretrained model(s) 323), the training data (which provides the examples from which to learn), and the learning algorithm (which dictates how the model adjusts its model parameters). The goal is for the model to capture the underlying patterns of the data without memorizing specific examples, thus enabling it to perform well on new, unseen data.

The model architecture is the specific arrangement and structure of the various components and/or layers that make up a model. In the context of a neural network, the model architecture may include the configuration of layers in the neural network, such as the number of layers, the type of layers (e.g., convolutional, recurrent, fully connected), the number of neurons in each layer, and the connections between these layers. In the context of a LLM comprised of a transformer architecture, which utilizes self-attention mechanisms to process and generate human-like text. The transformer model comprises an encoder-decoder structure, where the encoder processes the input text, and the decoder generates the output. The self-attention mechanism allows the model to weigh the importance of different words in a sentence, capturing long-range dependencies and contextual relationships. This architecture enables the model to handle large-scale data and understand complex language patterns. During training, the optimization algorithm such as Adam is used to minimize the loss function through backpropagation, and regularization techniques like dropout are employed to prevent overfitting, resulting in a robust and efficient language model capable of performing various natural language processing tasks such as predicting the ADR relations.

The model architecture also encompasses the choice and arrangement of features and algorithms used in various models, such as neural networks and transformers. The architecture determines how input data is processed and transformed through various computational steps to produce the output. The model architecture directly influences the model's ability to learn from the data effectively and efficiently, and it impacts how well the model performs tasks such as classification, regression, or prediction, adapting to the specific complexities and nuances of the data it is designed to handle.

The learning algorithm is the overall method or procedure used to adjust the model parameters 345 to fit the data. It dictates how the model learns from the data provided during training. This includes the steps or rules that the algorithm follows to process input data and make adjustments to the model's internal parameters (e.g., weights in neural networks) based on the output of the objective function. Examples of learning algorithms include gradient descent, backpropagation for neural networks, and splitting criteria in decision trees.

Various techniques may be employed by training and validation subsystem 315 to train machine learning models 330 using the learning algorithm, depending on the type of model and the specific task. For supervised learning models, where the training data includes both inputs and expected outputs (e.g., ground truth labels), gradient descent is a possible method. This technique iteratively adjusts the model parameters 345 to minimize or maximize an objective function (e.g., a loss function, a cost function, a contrastive loss function, etc.). The objective function is a method to measure how well the model's predictions match the actual labels or outcomes in the training data. It quantifies the error between predicted values and true values and presents this error as a single real number. The goal of training is to minimize this error, indicating that the model's predictions are, on average, close to the true data. Common examples of loss functions include mean squared error for regression tasks and cross-entropy loss for classification tasks.

The adjustment of the model parameters 345 is performed by the optimization function or algorithm, which refers to the specific method used to minimize (or maximize) the objective function. The optimization function is the engine behind the learning algorithm, guiding how the model parameters 345 are adjusted during training. It determines the strategy to use when searching for the best weights that minimize (or maximize) the objective function. Gradient descent is a primary example of an optimization algorithm, including its variants like stochastic gradient descent (SGD), mini-batch gradient descent, and advanced versions like Adam or RMSprop, which provide different ways to adjust learning rates or take advantage of the momentum of changes. For example, in training a neural network, backpropagation may be used with gradient descent to update the weights of the network based on the error rate obtained in the previous epoch (cycle through the full training dataset). Another technique in supervised learning is the use of decision trees, where a tree-like model of decisions is built by splitting the training dataset into subsets based on an attribute value test. This process is repeated on each derived subset in a recursive manner called recursive partitioning.

In unsupervised learning, where training data does not include labels, different techniques are used. Clustering is one method where data is grouped into clusters that maximize the similarities of data within the same cluster and maximize the differences with data in other clusters. The K-Means algorithm, for example, assigns each data point to the nearest cluster by minimizing the sum of distances between data points and their respective cluster centroids. Another technique, Principal Component Analysis (PCA), involves reducing the dimensionality of data by transforming it into a new set of variables, the principal components, which are uncorrelated and ordered so that the first few retain most of the variation present in all of the original variables. These techniques help uncover hidden structures or patterns in the data, which can be essential for feature reduction, anomaly detection, or preparing data for further supervised learning tasks.

Validating is another phase of developing machine learning models 330 where the model is checked for deficiencies in performance and the hyperparameters 340 are optimized based on validation data provided from the training and validation datasets 310. The validation data helps to evaluate the model's performance, such as accuracy, precision, recall, or F1-score, to gauge how well the model is likely to perform in real-world scenarios. Hyperparameter optimization, on the other hand, involves adjusting the settings that govern the model's learning process (e.g., learning rate, number of layers, size of the layers in neural networks) to find the combination that yields the best performance on the validation data. One optimization technique is grid search, where a set of predefined hyperparameter values are systematically evaluated. The model is trained with each combination of these values, and the combination that produces the best performance on the validation set is chosen. Although thorough, grid search can be computationally expensive and impractical when the hyperparameter space is large. A more efficient alternative optimization technique is random search, which samples hyperparameter combinations from a defined distribution randomly. This approach can in some instances find a good combination of hyperparameter values faster than grid search. Advanced methods like Bayesian optimization, genetic algorithms, and gradient-based optimization may also be used to find optimal hyperparameters more effectively. These techniques model the hyperparameter space and use statistical methods to intelligently explore the space, seeking hyperparameters that yield improvements in model performance.

An exemplary validation process includes iterative operations of inputting the validation subset of data into the trained algorithm(s) using a validation technique such as K-Fold Cross-Validation, Leave-one-out Cross-Validation, Leave-one-group-out Cross-Validation, Nested Cross-Validation, or the like, to fine-tune the hyperparameters and ultimately find the optimal set of hyperparameters. In some instances, a 5-fold cross-validation technique may be used to avoid overfitting the trained algorithm and/or to limit the number of selected features per split to the square-root of the total number of input features. In some instances, training dataset is split into 5 equal-size cohorts (or about equal-size), and every four of the cohorts are used to train an algorithm to generate five models (e.g, cohorts #1, 2, 3, and 4 are used to train and generate model 1, cohorts #1, 2, 3, and 5 are used to train and generate model 2, cohorts #1, 2, 4, and 5 are used to train and generate model 3, cohorts #1, 3, 4, and 5 are used to train and generate model 4, and cohorts #2, 3, 4 and 5 are used to train and generate model 5). Each model is evaluated (or validated) using the unused cohort in the training (e.g., for model 5, cohort #1 is used for validation). The overall performance of the training can be evaluated by an average performance of the five models. K-fold cross-validation provides a more robust estimate of a model's performance compared to a single training/validation split because it utilizes the entire dataset for both training and evaluation and reduces the variance in the performance estimate.

Once a machine learning model has been trained and validated, it undergoes a final evaluation using test data provided from the training and validation datasets 310, which is a separate subset of the data that has not been used during the training or validation phases. This step is crucial as it provides an unbiased assessment of the model's performance in simulating real-world operation. The test dataset serves as new, unseen data for the model, mimicking how the model would perform when deployed in actual use. During testing, the model's predictions are compared against the true values in the test dataset using various performance metrics such as accuracy, precision, recall, and mean squared error, depending on the nature of the problem (classification or regression). This process helps to verify the generalizability of the model-its ability to perform well across different data samples and environments-highlighting potential issues like overfitting or underfitting and ensuring that the model is robust and reliable for practical applications. The machine learning models 330 are fully validated and tested once the output predictions have been deemed acceptable by user defined acceptance parameters. Acceptance parameters may be determined using correlation techniques such as Bland-Altman method and the Spearman's rank correlation coefficients and calculating performance metrics such as the error, accuracy, precision, recall, receiver operating characteristic curve (ROC), etc.

### Inference Phase for Machine Learning Models

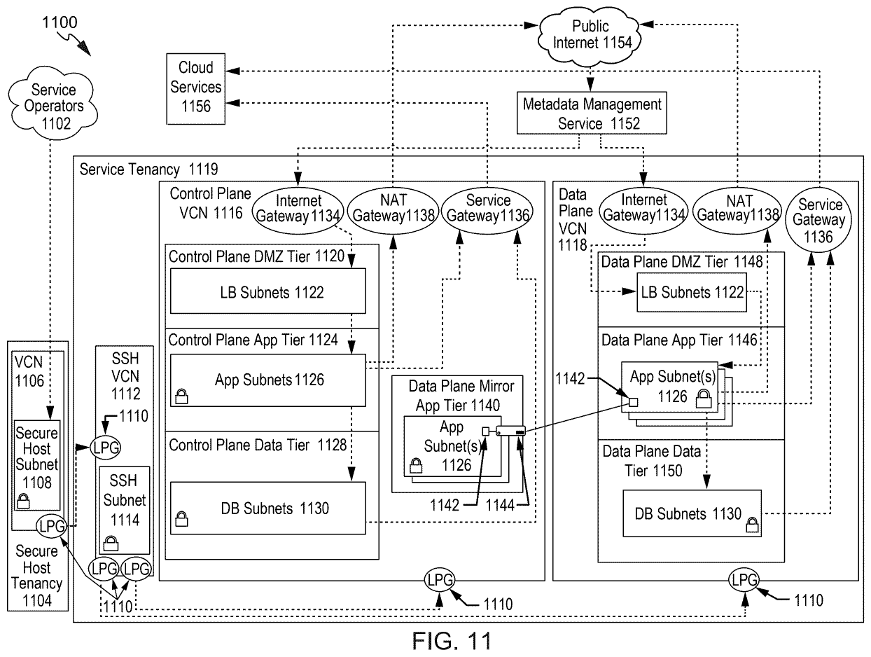

The inference subsystem 325 is comprised of various components for deploying the machine learning models 330 in a production environment (e.g., use as cloud service as described with respect to FIGS. 11-15). Deploying the machine learning models 330 includes moving the models from a development environment (e.g., the training and validation subsystem 315, where it has been trained, validated, and tested), into a production environment where it can make inferences on real-world data (e.g., input data 350). This step typically starts with the model being saved after training, including its parameters and configuration such as final architecture and hyperparameters. It is then converted, if necessary, into a format that is suitable for deployment, depending on the deployment environment. For instance, a model trained in a scientific computing environment such as Python might be converted into a Java-friendly format for integration into a larger enterprise application.

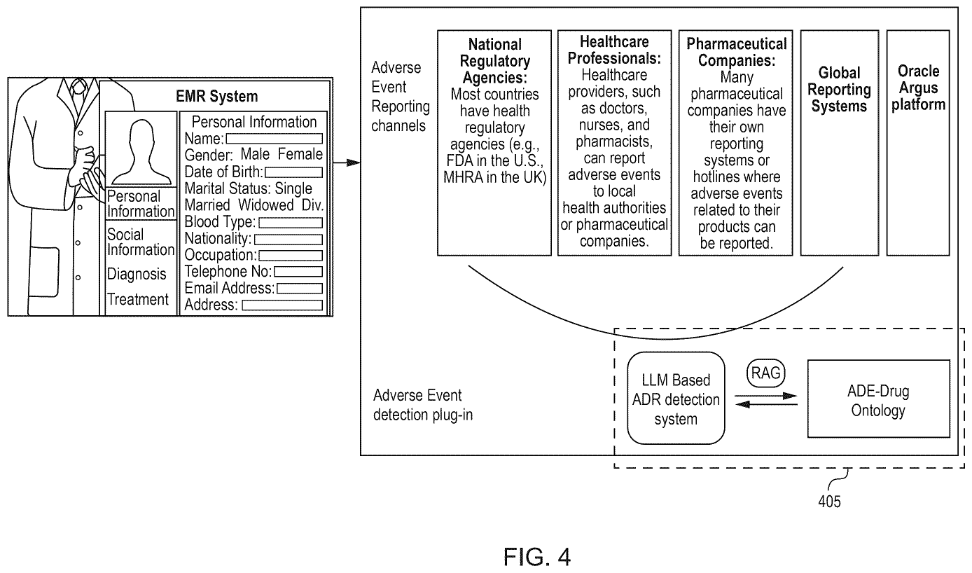

As shown in FIG. 4, one or more models can be deployed as part of a detection plug-in 405 for a larger enterprise application such as an EMR/EHR analyzer tool and/or ADR monitoring system for health care providers (e.g., medical centers, hospitals, nursing homes, etc.) for processing, analyzing, and reporting adverse event cases originating in premarket and post-market drugs, biologics, vaccines, devices, and combination products. Deployment can be conducted on various platforms, including on-premises servers or cloud environments like Oracle's Cloud Infrastructure (OCI), as described in greater detail with respect to FIGS. 11-15 (e.g., Oracle Argus Cloud Service is a component of the Oracle Safety Cloud, a simplified package of access, environment, and services in a subscription model).

Once deployed, the model is ready to receive input data 350 and return outputs (e.g., inferences 355). In some instances, the model resides as a component of a larger system or service (e.g., including additional downstream applications 335). In some instances, the models 330 and/or the inferences 355 can be used by the downstream applications 335 to provide further information. For example, the inferences 355 can be used to determine whether a patient had or is likely to have an ADR. The downstream applications can be configured to generate an output 360. In some instances, the output 360 comprises a report including inferences 355 and information generated by the downstream applications 335.

To manage and maintain its performance, a deployed model may be continuously monitored to ensure it performs as expected over time. This involves tracking the model's prediction accuracy, response times, and other operational metrics. Additionally, the model may require retraining or updates based on new data or changing conditions in the environment it is applied in. This can be useful because machine learning models can drift over time due to changes in the underlying data they are making predictions on-a phenomenon known as model drift. Therefore, maintaining a machine learning model in a production environment often involves setting up mechanisms for performance monitoring, regular evaluations against new test data, and potentially periodic updates and retraining of the model to ensure it remains effective and accurate in making predictions.

### Knowledge Graph Augmented ADR Prediction

Knowledge Graph (KG) in the form of bio-medical ontology can improve the performance of ADR prediction. A bio-medical ontology is comprised of controlled vocabularies that allow describing the meaning of data (its semantics) in a human and machine-readable way and may be used more to aid processing of information in biomedical research and in healthcare systems. These can fuse useful knowledge to LLM, which can lead to more accurate ADR prediction. This knowledge can be supplied in terms of the KG structure into the LLM during fine tuning (training) and inference phase. To supply the KG to the LLM in accordance with various embodiments, KG prompting techniques may be implemented that design a crafted prompt to convert structured KGs into text sequences, which in turn, can be fed as a context into the LLMs.

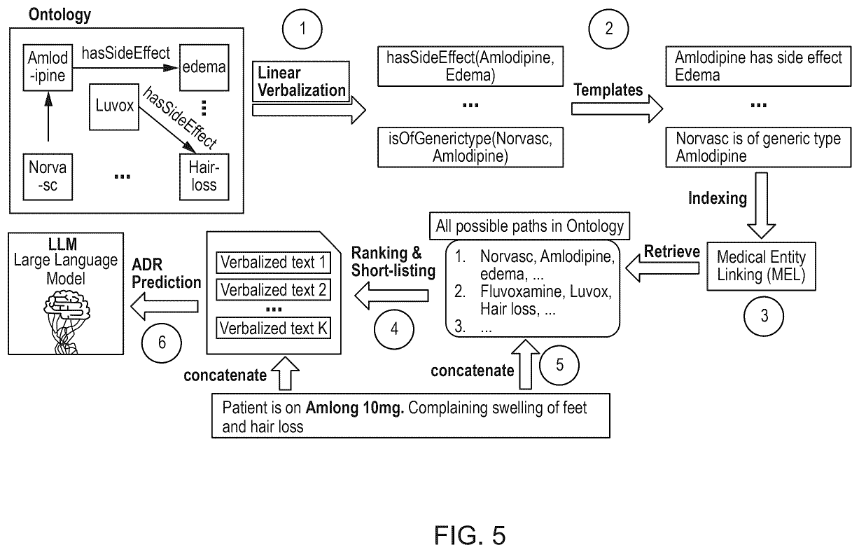

As shown in FIG. 5, the KG prompting techniques follow the steps below during training and inference phases (see explanation of said phases with respect to FIG. 3). For this explanation an ontology O is assumed to be comprised of a finite collection of concepts C={c₁, c₂, . . . , c_{N}}, relations T={t₁, t₂, . . . , t_{M}} and triplets of the form (cₖ, t, cₗ), where cₖ, cₗ∈C and t∈T. Also a path is a finite sequence of concepts, ρ: c₁, c₂, . . . , cₙ, where cᵢ, c_{{i+1}}∈C, t∈T, and (cᵢ, tᵢ, c_{{i+1}}) is a triplet, where 1≤i≤n.
1. Knowledge verbalization: As LLMs accept only textual input these cannot process factual triples of an ontology O which are represented over the symbolic graph. To map the symbolic fact from KGs to LLMs the triplet comprised of (c, t, c′); c, c′∈S, r∈T in the ontology O is transformed into its textual string, which is called verbalization. Although there are methods that particularly adopt or learn graph-to-text transformation, a linear verbalization is described herein for the KG prompting techniques.
2. For each triplet τ≡(c, t, c′) in the ontology, a template is generated which will represent the triplet in a textual form. Specifically for each relation t∈T a template is generated by which this triplet t can be suitably represented in the form of a textual description so that relevant knowledge can be better retrieved. A relation t is expanded a textual format, e.g, the relation instance, part_of (Urinary Bladder, Female Pelvis) is expanded as: “Urinary Bladder is part of Female Pelvis. This is the verbalized form of the triplet, verbalized(c, t, c′). These triplets are indexed in some arbitrary order.
3. Entity-specific knowledge retrieval: Triplets are retrieved in the ontology corresponding to a pair of entities appearing in a triple

$$τ ≡ ( s , r , {s}^{′} ) ∈ {Y}_{R}^{S} .$$

The subject span s∈S with a mapped entity type e∈E is linked to the most similar concept c in the ontology O (which is a medical entity linking problem (MEL). Similarly, the object span s′∈S with a mapped entity type e′∈E, is linked to the most similar concept c′ in the ontology O. Now all possible paths between c and c′ in the ontology O, are identified where the total number of paths is Γ.
4. Ranking of paths: The T paths are then ranked using one or more ranking methods such as use of a pre-trained cross-encoder, bi-encoder, competition ranker, or the like. Specifically, each path ρ is verbalized by concatenating verbalization of each triplets with “:”—concatenation operator, that is ρ is written as −verbalized(c₁, t₁, c₂); verbalized(c₂, t₂, c₃); . . . ; verbalized(c_{{n-1}}, t_{{n-1}}, cₙ).
5. Then each input to the pre-trained cross-encoder will be the concatenation of pair of spans (subject and object spans) with the verbalized form of each path. The cross-encoder will output a scalar value between 0 to 1 for each such input. These scalar values are then used as ranking scores for Γ paths.
6. LLM reasoning: Once the ranking is completed, the top K top paths (above) are chosen, and concatenate along with the pair of input spans, which are appended after the ADR task description in the prompt body (see Table. 1). During training or inference phase, the prompting remains identical.

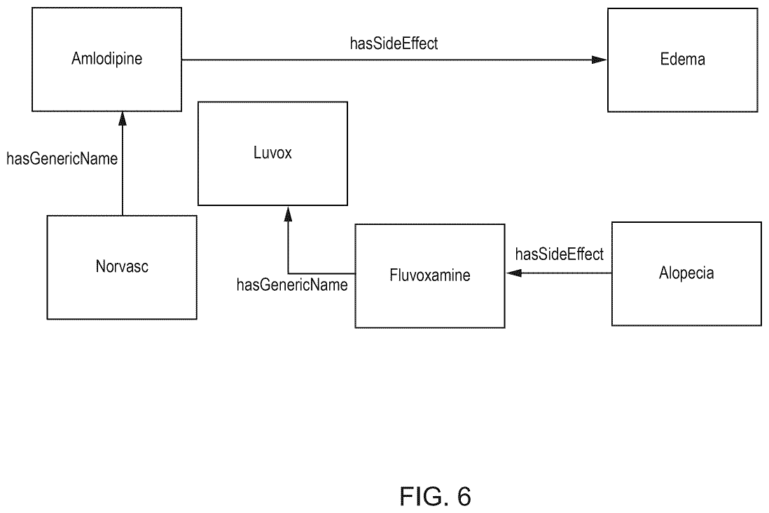

These K top paths are added as part of prompt as verbalized text. For illustration purposes, consider the toy Ontology of Adverse Drug Reaction in FIG. 6. For a complete workflow of this pipeline (KG→Linear Verbalization→Templatization→Entity-specific knowledge retrieval (Indexing)→Generation of all paths in Ontology→Ranking) refer to FIG. 5. Using this ontology, the prompt may be modified for the input text (see Table. 2). Note the span “Amlong” is mapped to the concept ‘Norvasc’ in the ontology, and the span “hair loss” is mapped to the concept ‘Edema’ in the ontology.

TABLE 2
Modified Prompts using knowledge of Ontology in FIG. 6 for ADR Prediction

| | |
|---|---|
| Prompt | You are ChatPharmacovigilance, an expert ADR detection BOT. Your task is |
| | to identify Adverse drug reaction from adr text. |
| | We consider two entity types: <Drug> and <ADE>. The entity type Drug |
| | corresponds to Medicine name. |
| | The entity type Adverse Drug Event (ADE) is a drug event or a drug reaction |
| | which is an unintended reaction or |
| | event suspected to be due to the routine use of drugs under normal |
| | conditions. |
| | 1. You need to assign a causation of drug to ADE by predicting a relation |
| | between Drug and ADE. For that we introduce two types of relations between |
| | entity types Drug and ADE, they are <ADR> (Adverse Durg Reaction) and |
| | <No-ADR>. |
| | 2. Consider the spans in the input text that are mapped to entity type Drug (or |
| | Medicine Name) or DE. |
| | 3. If you find the relation ADR between the span mapped to Drug and the span |
| | mapped to DE then respond with relation ADR, |
| | else respond with relation No-ADR. |
| | Format your response with JSON objects with “Relation”, “Span of Drug” and |
| | “Span of ADE’ as the keys. |
| | If the information is not available use “unknown” as the value. |
| | Make your response as short as possible.\ |
| | adr text: ″′{ adr_text}′″ |
| | adr_text = <Patient is on Amlong 10 mg. Complaining of swelling of feet and |
| | hair loss> |
| | { |
| | “Span of Drug”: “Amlong” |
| | “Span of ADE”: “swelling of feet” |
| | “Span of ADE”: “hair loss” |
| | } |
| | Use The following information for prediction of ADR and No-ADR relations. |
| | { |
| | <“Span of Drug”: “Amlong” |
| | “Span of ADE”: “swelling of feet” |
| | ′Amlong’ is mapped to: ′Norvasc’, |
| | ′Swelling of feet’ is mapped to: ′edema’; |
| | (Norvasc is generic name of Amlodipine; Amlodipine has side effect of |
| | edema)> |
| | } |

### Experimental Efforts and Examples

Datasets: For experimentation purposes the Adverse Drug Effect (ADE dataset described in Gurulingappa, H., Rajput, A. M., Roberts, A., Fluck, J., Hofmann-Apitius, M., Toldo, L.: Development of a benchmark corpus to support the automatic extraction of drug-related adverse effects from medical case reports. J. Biomed. Informatics 45(5), 885-892 (2012)) was used. The ADE dataset contains 4271 unique labelled text samples, with a total of 6821 drug-ADE relation tuples appearing in those texts. These are the positive relations that are considered for training. Negative samples were also prepared as these are expected to increase the performance of the model on unseen data. This dataset also comprises of 16,695 labeled negative sentences; however, the Drug-ADE tuples are not marked. Some of the examples were filtered out because it was not possible to extract the tuples. Further the experiments were designed to consider not only the negative samples provided, but also add more samples to this collection, where possible pairs of both drug and ADE may not be present. These consolidated negative samples are simply all the tuples of drug-ADE which are not annotated with ADR relation in the original dataset. Overall, there are 4271 positive relations and 1663 negative relations present in the data. This data was then split in ratio of 80:20 to produce the training and test sets. This was then used for LLM fine tuning.

These K top paths are added as part of prompt as verbalized text. For illustration purposes, consider the example Ontology of Adverse Drug Reaction in FIG. 6. For a complete workflow of this pipeline (KG→Linear Verbalization→Templatization→Entity-specific knowledge retrieval (Indexing)→Generation of all paths in Ontology→Ranking) see FIG. 5. Using this ontology, the prompt may be modified for the input text (see Table. 2). Note the span “Amlong” is mapped to the concept ‘Norvasc’ in the ontology, and the span “hair loss” is mapped to the concept ‘Edema’ in the ontology.

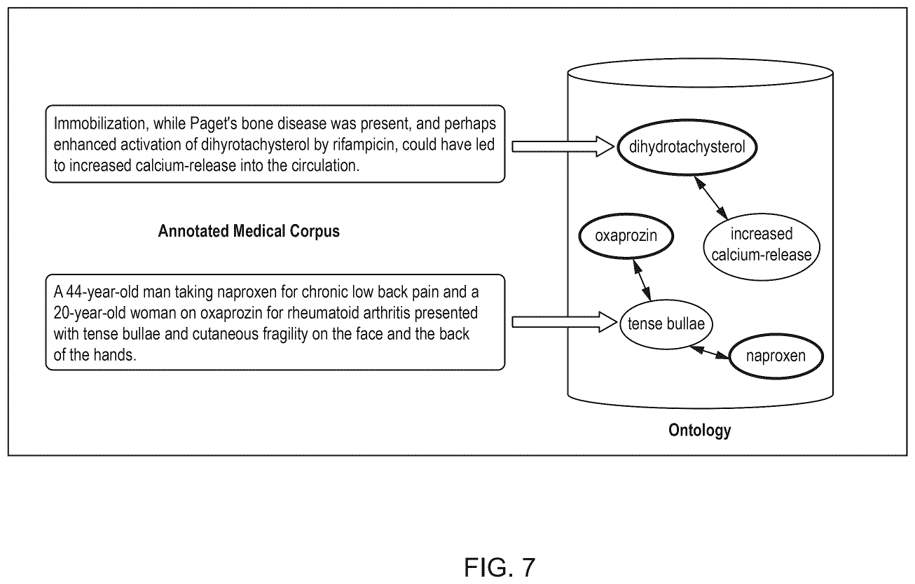

Ontology: In the experiments, a base ontology was created from a collection of clinical narratives on ADE. Drug and Event were annotated in the text-corpus (with the help of an LLM). Linkage between all Drug-Events were created to create an ADR ontology. For example, the following steps were used with reference to FIG. 7 (two clinical text were considered). Drug names and events were annotated in the text using a separate LLM for the texts where ADR is positive. Then nodes were created for each detected drug and event in the ontology. In the one text, two spans were picked up and connected: dihydrotachysterol (mapped to Drug) and increased calcium release (mapped to ADE). In another text, spans: naproxen (mapped to Drug), oxaprozin (mapped to Drug) and tense bullae (mapped to ADE) were picked up and connected. Links were provided between spans mapped to Drug and the span mapped to ADE.

LLM details: For the pretrained model, Mistral-7B-Instruct-v0.3 Large Language Model (LLM) was used, which is an instruct fine-tuned version of the Mistral-7B-v0.3. The main parameters with the best settings are shown in Table 3. Parameter-efficient fine tuning was performed with LORA.

TABLE 3
Hyperparameter Settings:

| Task | Hyper-parameter | Value |
|---|---|---|
| Basic | bf16 | True |
| | batch size | 64 |
| | Learning_rate | 2.0e−05 |
| | lr_scheduler_type | “cosine” |
| LORA | r | 64 |
| PEFT | lora_alpha | 16 |
| | lora_dropout | 0.1 |
| Generation | max_new_tokens | 1024 |
| | do_sample | True |
| | temperature | 0.1 |

RAG-Architecture details: The RAG architecture is an approach that combines the strengths of retrieval-based and generation-based models to enhance the performance of language tasks. In the RAG model, the system first retrieves relevant documents or passages from a large corpus of text such as EHR records using a retrieval mechanism, such as a dense passage retriever or a traditional search algorithm. These retrieved documents or passages provide additional context and information that can be utilized by the generation component of the model, a Transformer-based language model such as Mistral-7B-Instruct-v0.3. The generation model then synthesizes this retrieved information with the input prompt to produce a more accurate and contextually enriched output. This hybrid approach leverages the vast knowledge embedded in the retrieval corpus and the sophisticated language generation capabilities of LLMs, resulting in more informed and precise responses, especially for tasks requiring detailed or factual information.

More specifically, the RAG architecture was used to infuse knowledge into the pretrained LLM by selecting concepts from ontology through entity linking. with traditional information retrieval systems. That is, spans mapped to Drug and ADEs were linked to concepts in the appropriate ontology using entity linking. For that embedding vectors for Drug and ADE spans were generated using a separate LLM and they were stored in a Vector database Qdrant. For the identified concepts corresponding to Drug/ADEs in ontology, all paths in the Vector database were identified using approximate nearest neighbor (ANN) search for faster retrieval. Then top 3 matches (configurable) were injected as part of prompt to the pretrained LLM to make ADR predictions. Since a base ontology was used, there was no information added for No-ADR relations from the ontology as part on instruction prompting.

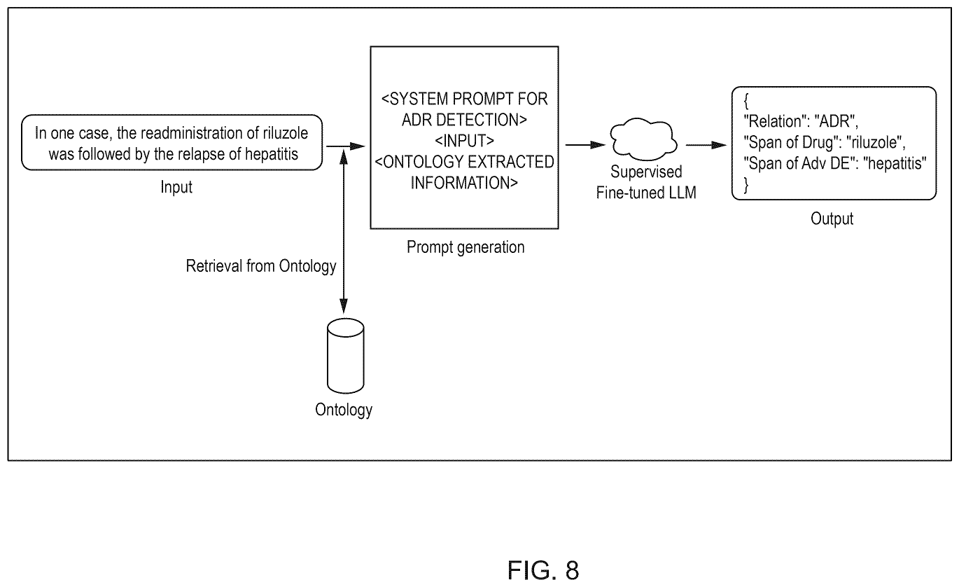

A schematic diagram of implementation of RAG-assisted ADE prediction on fine-tuned LLM is illustrated in FIG. 8 (see discussion of FIGS. 1-5 for additional details). The steps for infusing knowledge into LLM by selecting concepts from ontology include:
Annotating spans in medical text for drug and ADE entities to create labelled medical text
Link spans mapped to Drug and ADEs to concepts in the appropriate ontology using entity linking. The appropriate ontology can be identified by running a search (using the drug and ADE entities) on ontology data (ontologies covering various drug and ADE entities) in a database and/or running searches on medical literature and generating the ontology as described herein.
Generate, using a separate LLM, embedding vectors for the one or more triplets in the ontology to represent each of the one or more triplets in form of a textual description using and store them in vector database such as Qdrant.
For the identified concepts corresponding to Drug/ADEs in ontology, identify all paths in the vector database using a search such as an ANN search for faster retrieval.
Then inject top matches (configurable) as part of prompt to the fine-tuned LLM to make ADR prediction.

Metrics for experimentation: Three metrics were used for finding the efficacy of different approaches: precision, recall and F1-score. In pharmacovigilance (PV) literature, recall is suggested as primary metric for solutions detecting adverse drug reaction in a medical text as the primary goal of PV is to ensure patient safety by identifying and mitigating risks associated with drug use.

Results: This discussion is separated into three parts, zero-shot ADE prediction, post fine tuning ADE prediction and bench marking with existing approaches on ADE prediction. The Precision, Recall and F1 score are reported for this task, where ADR relation or No-ADR relation are predicted between spans reflecting Drug and AE.

Zero-shot ADE prediction: In zero shot setting an LLM Is prompted to predict ADE without any examples, thereby taking advantage of the reasoning patterns it has gleaned. However, additionally supplementary prompts may also be fed to the LLM. Considering both cases, inference without the knowledge of ontology and ontology-augmented inference. The results are shown in Table 4. Note that F1 score (gain of 13%), and importantly recall improve with the fusion of ontology into the LLM.

TABLE 4
Zero shot and Instruction-tuned inference on LLM:

| Training | Inference | | | F-1 | |
|---|---|---|---|---|---|
| type | type | Precision | Recall | score | Accuracy |
| Zero-shot | wo Ontology | 0.71 | 0.85 | 0.77 | 0.72 |
| learing | with | 0.81 | 0.94 | 0.87 | 0.8 |
| | Ontology | | | | |
| Instruction | wo Ontology | 0.76 | 0.98 | 0.85 | 0.82 |
| tuning | with | 0.85 | 0.99 | 0.90 | 0.89 |
| | Ontology | | | | |

Inference on instruction fine-tuned LLM: LLMs were trained using (instruction, output) pairs, where the instructions are created using both positive and negative instances of ADE relation on datasets, and output mainly captures the affirmative or the negative response on the corresponding instances. Again, considering both the cases, inference without the knowledge of ontology and ontology-guided inference. The results are shown in Table 4. In this case also, recall and F1 score increase (raise of 7%) with the fusion of ontology during inference.

By observation of these results it is possible to conclude that instruction fine tuning indeed leads to improvement in performance of ADE prediction, this also highlights the fact that an LLM equipped with knowledge inside the input text exhibits better performance. It is also possible to conclude that ontology indeed leads to improvement of performance of ADE prediction as witnessed in both the cases for zero shot inference and post instruction fine tuning inference (see Table 4).

Bench marking with existing methods on ADE datasets: The performance of the models was also compared with a few baseline models on ADE datasets (see Table 5), RECON model for relation prediction using knowledge graph context (Bastos, A., Nadgeri, A., Singh, K., Mulang, I. O., Shekarpour, S., Hoffart, J., Kaul, M.: Recon: Relation extraction using knowledge graph context in a graph neural network. In: Proceedings of the Web Conference '21. p. 1673-1685. WWW '21 (2021)), CRNN model which uses deep neural network (Huynh, T., He, Y., Willis, A., Rüger, S. M.: Adverse drug reaction classification with deep neural networks. In: 26th COLING '16, Proceedings of the Conference. pp. 877-887. ACL (2016)), T5 architecture (Raffel, C., Shazeer, N., Roberts, A., Lee, K., Narang, S., Matena, M., Zhou, Y., Li, W., Liu, P. J.: Exploring the limits of transfer learning with a unified text-to-text transformer. J. Mach. Learn. Res. 21(1) (2020)), CNN-embedding-based model (Rawat, A., Wani, M. A., ElAffendi, M., Imran, A. S., Kastrati, Z., Daudpota, S. M.: Drug adverse event detection using text-based convolutional neural networks (TextCNN) technique. Electronics 11(20)(2022)) and ReOnto (Jain, M., Singh, K., Mutharaju, R.: Reonto: A neuro-symbolic approach for biomedical relation extraction. In: Machine Learning and Knowledge Discovery in Databases: Research Track-ECML PKDD '23, Proceedings, Part IV. LNCS, vol. 14172, pp. 230-247. Springer (2023)). These models were trained and tested on ADE dataset in the work of (Jain, M., Singh, K., Mutharaju, R.: Reonto: A neuro-symbolic approach for biomedical relation extraction. In: Machine Learning and Knowledge Discovery in Databases: Research Track-ECML PKDD '23, Proceedings, Part IV. LNCS, vol. 14172, pp. 230-247. Springer (2023)). The results from that paper are provided and the LLM-generated results in accordance with embodiments of the present disclosure are appended for comparing, however only the F1 score is reflected. It is observable that LLM-based ADE prediction performs nearly as good as most of the other neural-based models, as it captures context information in the model through ontology, although it cannot outperform RECON and ReOnto. However, with an LLM-guided ADE prediction very little analysis of data is needed compared to RECON and ReOnto.

TABLE 5
Inference on instruction fine-tuned LLM on ADE data set

| | F-1 |
|---|---|
| Model | score |
| CRNN [HuynhHWR16] | 0.87 |
| RECON [Bastos + 21] | 0.92 |
| CNN-embedding [Rawat + 22] | 0.89 |
| ReOnto [JainSM23] | 0.96 |
| Zero-shot LLM inference without Ontology (present disclosure) | 0.87 |
| Fine-tuned LLM inference with Ontology (present disclosure) | 0.90 |

### Ablation Study

An ablation study was performed to help understand the performance of LLM-guided ADE prediction. As demonstrated above, adding knowledge via ontology improves the performance of the model. Additionally, the effectiveness of supplementary prompts used as part of in-context learning was investigated as part of the ablation study. The prompt texts were selected by observing the samples (both positive and negative) and extracting clues for reasoning paths. Finding these clues for writing prompts is important as it affects the performance of the model. This is especially true for predicting the negative instances of ADE prediction, i.e., No-ADR relations.

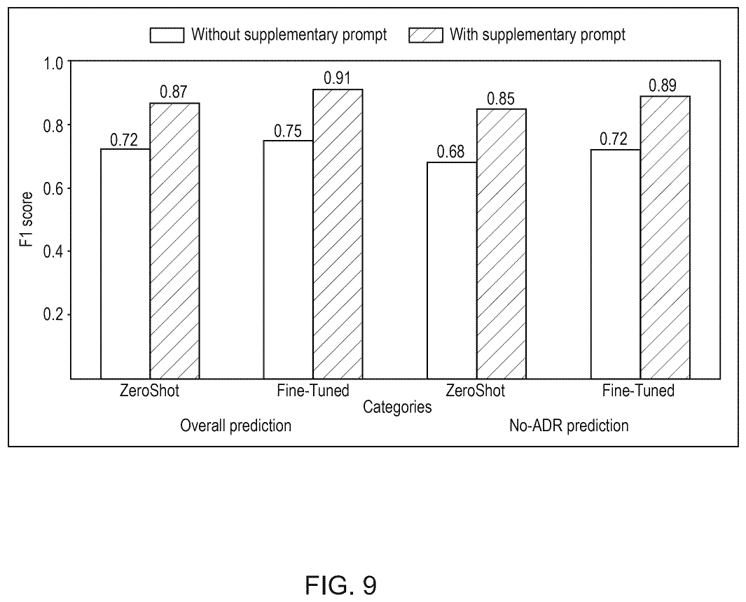

These observations are captured in FIG. 9. The performance improves as more and more meaningful reasoning paths are provided to the LLM in the form of prompts. However, it is very difficult to quantify all intermediate improvements. This is the reason performance was measured for two cases,—that is without any supplementary prompt and with supplementary prompt. In case of overall prediction there is appreciable improvement of F1 score both for zero shot learning (17%) and fine tuning (21%). This improvement is more pronounced in case of prediction of No-ADR relation, the improvement varies from 25% to 37%. This implies one needs to provide adequate reasoning paths based on dataset in terms of prompts for predicting negative instances. This also shows the importance of properly capturing hidden domain knowledge inside datasets (albeit heuristically).

### Techniques for Predicting ADR

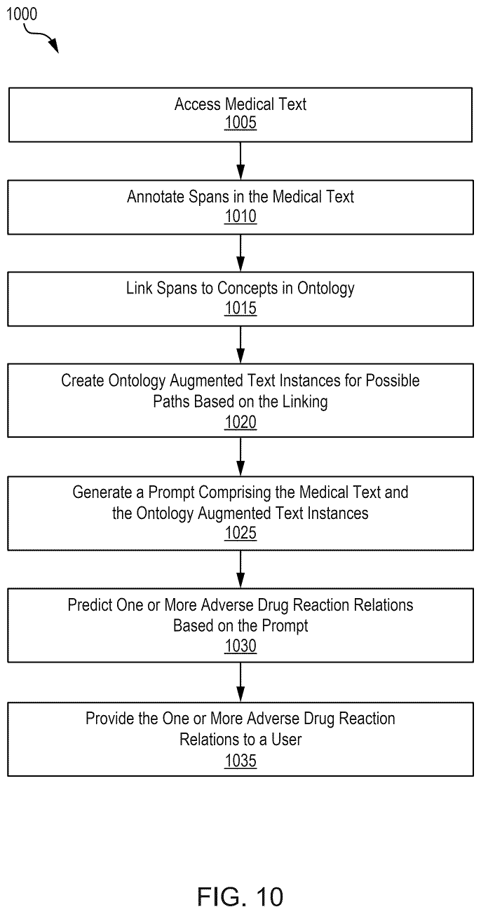

FIG. 10 depicts a simplified flowchart 1000 depicting a process for predicting ADR implemented by the ADR-LLM framework according to certain embodiments. The processing depicted in FIG. 10 may be implemented in software (e.g., code, instructions, program) executed by one or more processing units (e.g., processors, cores) of the respective systems, hardware, or combinations thereof. The software may be stored on a non-transitory storage medium (e.g., on a memory device). The method presented in FIG. 10 and described below is intended to be illustrative and non-limiting. Although FIG. 10 depicts the various processing steps occurring in a particular sequence or order, this is not intended to be limiting. In certain alternative embodiments, the steps may be performed in some different order or some steps may also be performed in parallel.

The process commences in step 1005, where medical text associated with a patient is accessed.

At step 1010, spans in the medical text are annotated for drug and event entities to create labelled medical text.

At step 1015, the spans in the labelled medical text are linked using entity linking to concepts in an ontology. The linking comprises: for a subject span in the labelled medical text with a mapped entity type of drug or event, link the subject span to a most similar subject concept in the ontology, and for an object span in the labelled medical text with a mapped entity type of drug or event, link the object span to a most similar object concept in the ontology.

In some instances, the ontology comprises concepts, relations, and one or more triplets having a subject concept, a relation, and an object concept, and a path in the ontology is a sequence of concepts and relations identified therein by associated one or more triplets.

In some instances, the process further comprises: retrieving, based on a query of ontology data, an ontology associated with the medical text; verbalizing, using one or more templates, the one or more triplets in the ontology to represent each of the one or more triplets in form of a textual description; embedding the textual description for each of the one or more triplets; and storing the embedded textual description for each of the one or more triplets in a vector database. The linking is performed between the spans in the labelled medical text to the concepts in the embedded textual description for each of the one or more triplets stored in the vector database.

In some instances, a determination of whether a subject span or an object span in the labelled medical text with the mapped entity type of drug or event is most similar to a corresponding subject concept or object concept in the ontology is performed using an approximate nearest neighbor (ANN) search, and the ANN search performs a semantic similarity search in the vector database using the subject spans and the object spans as query data points.

In some instances, ranking each of the possible paths in the ontology comprises: verbalizing each of the identified possible paths in the ontology by concatenating the textual description for the one or more triplets associated with each of the identified possible paths; concatenating each pair of the linked subject and object spans with the associated verbalized form of each of the identified possible paths to generate a corresponding possible path input; determining, by a pre-trained cross-encoder, a scaler value for each possible path input; and ranking the possible path inputs based on the corresponding scalar value for each possible path input, wherein the top number of paths are identified from all the possible paths based on the ranking of the possible path inputs.

At step 1020, for each pair of linked subject and object spans: possible paths (e.g., all possible paths) in the ontology are identified between the subject concept and the object concept, each of the possible paths in the ontology are ranked, a top number of paths from all the possible paths are identified based on the ranking, and a verbalized form of each path of the top number of paths is concatenated with the subject pan and the object span from the labelled medical text to create an ontology augmented text instance corresponding to the pair of linked subject and object spans.

At step 1025, a prompt is generated comprising the medical text and each ontology augmented text instance.

In some instances, the prompt further comprises an overview of the adverse drug reaction relation task and intermediate texts as part of in-context learning.

At step 1030, one or more adverse drug reaction relation predictions are generated by a generative artificial intelligence model for the patient based on the prompt.

In some instances, the generative artificial intelligence model is a LLM fine-tuned for the adverse drug reaction relation task.

At step 1035, the one or more adverse drug reaction relation predictions are provided to a user.

In some instances, the one or more adverse drug reaction relation predictions are provided to a user as part of plug-in for an application associated with a cloud service.

### Illustrative Systems

The ADR-LLM framework and techniques described herein can be offered as a cloud computing service. For example, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 11 is a block diagram 1100 illustrating an example pattern of an IaaS architecture, according to at least one embodiment. Service operators 1102 can be communicatively coupled to a secure host tenancy 1104 that can include a virtual cloud network (VCN) 1106 and a secure host subnet 1108. In some examples, the service operators 1102 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 1106 and/or the Internet.

The VCN 1106 can include a local peering gateway (LPG) 1110 that can be communicatively coupled to a secure shell (SSH) VCN 1112 via an LPG 1110 contained in the SSH VCN 1112. The SSH VCN 1112 can include an SSH subnet 1114, and the SSH VCN 1112 can be communicatively coupled to a control plane VCN 1116 via the LPG 1110 contained in the control plane VCN 1116. Also, the SSH VCN 1112 can be communicatively coupled to a data plane VCN 1118 via an LPG 1110. The control plane VCN 1116 and the data plane VCN 1118 can be contained in a service tenancy 1119 that can be owned and/or operated by the IaaS provider.

The control plane VCN 1116 can include a control plane demilitarized zone (DMZ) tier 1120 that acts as a perimeter network (e.g., portions of a corporate network between the corporate intranet and external networks). The DMZ-based servers may have restricted responsibilities and help keep breaches contained. Additionally, the DMZ tier 1120 can include one or more load balancer (LB) subnet(s) 1122, a control plane app tier 1124 that can include app subnet(s) 1126, a control plane data tier 1128 that can include database (DB) subnet(s) 1130 (e.g., frontend DB subnet(s) and/or backend DB subnet(s)). The LB subnet(s) 1122 contained in the control plane DMZ tier 1120 can be communicatively coupled to the app subnet(s) 1126 contained in the control plane app tier 1124 and an Internet gateway 1134 that can be contained in the control plane VCN 1116, and the app subnet(s) 1126 can be communicatively coupled to the DB subnet(s) 1130 contained in the control plane data tier 1128 and a service gateway 1136 and a network address translation (NAT) gateway 1138. The control plane VCN 1116 can include the service gateway 1136 and the NAT gateway 1138.

The control plane VCN 1116 can include a data plane mirror app tier 1140 that can include app subnet(s) 1126. The app subnet(s) 1126 contained in the data plane mirror app tier 1140 can include a virtual network interface controller (VNIC) 1142 that can execute a compute instance 1144. The compute instance 1144 can communicatively couple the app subnet(s) 1126 of the data plane mirror app tier 1140 to app subnet(s) 1126 that can be contained in a data plane app tier 1146.

The data plane VCN 1118 can include the data plane app tier 1146, a data plane DMZ tier 1148, and a data plane data tier 1150. The data plane DMZ tier 1148 can include LB subnet(s) 1122 that can be communicatively coupled to the app subnet(s) 1126 of the data plane app tier 1146 and the Internet gateway 1134 of the data plane VCN 1118. The app subnet(s) 1126 can be communicatively coupled to the service gateway 1136 of the data plane VCN 1118 and the NAT gateway 1138 of the data plane VCN 1118. The data plane data tier 1150 can also include the DB subnet(s) 1130 that can be communicatively coupled to the app subnet(s) 1126 of the data plane app tier 1146.

The Internet gateway 1134 of the control plane VCN 1116 and of the data plane VCN 1118 can be communicatively coupled to a metadata management service 1152 that can be communicatively coupled to public Internet 1154. Public Internet 1154 can be communicatively coupled to the NAT gateway 1138 of the control plane VCN 1116 and of the data plane VCN 1118. The service gateway 1136 of the control plane VCN 1116 and of the data plane VCN 1118 can be communicatively coupled to cloud services 1156.

In some examples, the service gateway 1136 of the control plane VCN 1116 or of the data plane VCN 1118 can make application programming interface (API) calls to cloud services 1156 without going through public Internet 1154. The API calls to cloud services 1156 from the service gateway 1136 can be one-way: the service gateway 1136 can make API calls to cloud services 1156, and cloud services 1156 can send requested data to the service gateway 1136. But, cloud services 1156 may not initiate API calls to the service gateway 1136.

In some examples, the secure host tenancy 1104 can be directly connected to the service tenancy 1119, which may be otherwise isolated. The secure host subnet 1108 can communicate with the SSH subnet 1114 through an LPG 1110 that may enable two-way communication over an otherwise isolated system. Connecting the secure host subnet 1108 to the SSH subnet 1114 may give the secure host subnet 1108 access to other entities within the service tenancy 1119.

The control plane VCN 1116 may allow users of the service tenancy 1119 to set up or otherwise provision desired resources. Desired resources provisioned in the control plane VCN 1116 may be deployed or otherwise used in the data plane VCN 1118. In some examples, the control plane VCN 1116 can be isolated from the data plane VCN 1118, and the data plane mirror app tier 1140 of the control plane VCN 1116 can communicate with the data plane app tier 1146 of the data plane VCN 1118 via VNICs 1142 that can be contained in the data plane mirror app tier 1140 and the data plane app tier 1146.

In some examples, users of the system, or customers, can make requests, for example create, read, update, or delete (CRUD) operations, through public Internet 1154 that can communicate the requests to the metadata management service 1152. The metadata management service 1152 can communicate the request to the control plane VCN 1116 through the Internet gateway 1134. The request can be received by the LB subnet(s) 1122 contained in the control plane DMZ tier 1120. The LB subnet(s) 1122 may determine that the request is valid, and in response to this determination, the LB subnet(s) 1122 can transmit the request to app subnet(s) 1126 contained in the control plane app tier 1124. If the request is validated and requires a call to public Internet 1154, the call to public Internet 1154 may be transmitted to the NAT gateway 1138 that can make the call to public Internet 1154. Metadata that may be desired to be stored by the request can be stored in the DB subnet(s) 1130.

In some examples, the data plane mirror app tier 1140 can facilitate direct communication between the control plane VCN 1116 and the data plane VCN 1118. For example, changes, updates, or other suitable modifications to configuration may be desired to be applied to the resources contained in the data plane VCN 1118. Via a VNIC 1142, the control plane VCN 1116 can directly communicate with, and can thereby execute the changes, updates, or other suitable modifications to configuration to, resources contained in the data plane VCN 1118.

In some embodiments, the control plane VCN 1116 and the data plane VCN 1118 can be contained in the service tenancy 1119. In this case, the user, or the customer, of the system may not own or operate either the control plane VCN 1116 or the data plane VCN 1118. Instead, the IaaS provider may own or operate the control plane VCN 1116 and the data plane VCN 1118, both of which may be contained in the service tenancy 1119. This embodiment can enable isolation of networks that may prevent users or customers from interacting with other users', or other customers', resources. Also, this embodiment may allow users or customers of the system to store databases privately without needing to rely on public Internet 1154, which may not have a desired level of threat prevention, for storage.

In other embodiments, the LB subnet(s) 1122 contained in the control plane VCN 1116 can be configured to receive a signal from the service gateway 1136. In this embodiment, the control plane VCN 1116 and the data plane VCN 1118 may be configured to be called by a customer of the IaaS provider without calling public Internet 1154. Customers of the IaaS provider may desire this embodiment since database(s) that the customers use may be controlled by the IaaS provider and may be stored on the service tenancy 1119, which may be isolated from public Internet 1154.

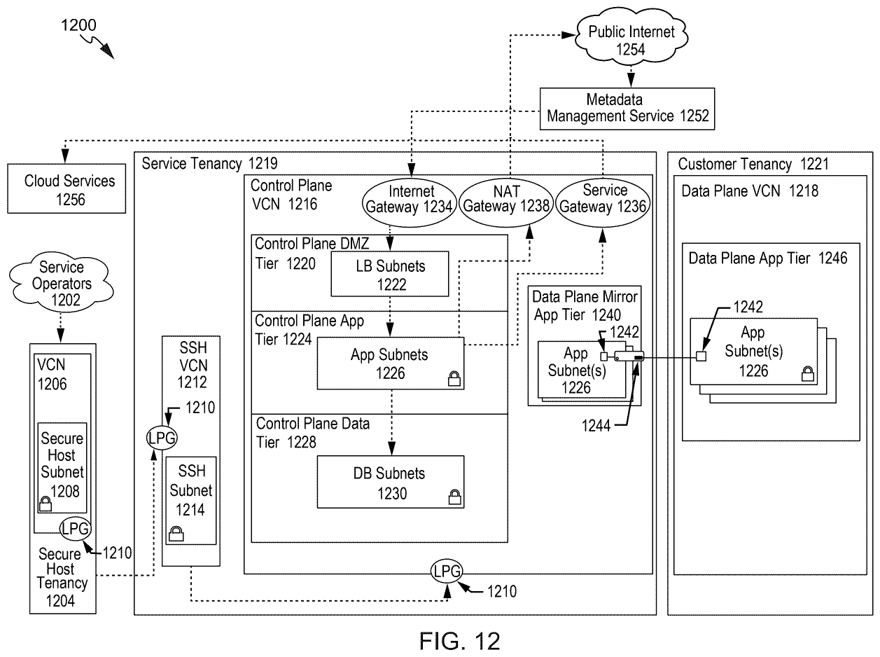

FIG. 12 is a block diagram 1200 illustrating another example pattern of an IaaS architecture, according to at least one embodiment. Service operators 1202 (e.g., service operators 1102 of FIG. 11) can be communicatively coupled to a secure host tenancy 1204 (e.g., the secure host tenancy 1104 of FIG. 11) that can include a virtual cloud network (VCN) 1206 (e.g., the VCN 1106 of FIG. 11) and a secure host subnet 1208 (e.g., the secure host subnet 1108 of FIG. 11). The VCN 1206 can include a local peering gateway (LPG) 1210 (e.g., the LPG 1110 of FIG. 11) that can be communicatively coupled to a secure shell (SSH) VCN 1212 (e.g., the SSH VCN 1112 of FIG. 11) via an LPG 1110 contained in the SSH VCN 1212. The SSH VCN 1212 can include an SSH subnet 1214 (e.g., the SSH subnet 1114 of FIG. 11), and the SSH VCN 1212 can be communicatively coupled to a control plane VCN 1216 (e.g., the control plane VCN 1116 of FIG. 11) via an LPG 1210 contained in the control plane VCN 1216. The control plane VCN 1216 can be contained in a service tenancy 1219 (e.g., the service tenancy 1119 of FIG. 11), and the data plane VCN 1218 (e.g., the data plane VCN 1118 of FIG. 11) can be contained in a customer tenancy 1221 that may be owned or operated by users, or customers, of the system.

The control plane VCN 1216 can include a control plane DMZ tier 1220 (e.g., the control plane DMZ tier 1120 of FIG. 11) that can include LB subnet(s) 1222 (e.g., LB subnet(s) 1122 of FIG. 11), a control plane app tier 1224 (e.g., the control plane app tier 1124 of FIG. 11) that can include app subnet(s) 1226 (e.g., app subnet(s) 1126 of FIG. 11), a control plane data tier 1228 (e.g., the control plane data tier 1128 of FIG. 11) that can include database (DB) subnet(s) 1230 (e.g., similar to DB subnet(s) 1130 of FIG. 11). The LB subnet(s) 1222 contained in the control plane DMZ tier 1220 can be communicatively coupled to the app subnet(s) 1226 contained in the control plane app tier 1224 and an Internet gateway 1234 (e.g., the Internet gateway 1134 of FIG. 11) that can be contained in the control plane VCN 1216, and the app subnet(s) 1226 can be communicatively coupled to the DB subnet(s) 1230 contained in the control plane data tier 1228 and a service gateway 1236 (e.g., the service gateway 1136 of FIG. 11) and a network address translation (NAT) gateway 1238 (e.g., the NAT gateway 1138 of FIG. 11). The control plane VCN 1216 can include the service gateway 1236 and the NAT gateway 1238.

The control plane VCN 1216 can include a data plane mirror app tier 1240 (e.g., the data plane mirror app tier 1140 of FIG. 11) that can include app subnet(s) 1226. The app subnet(s) 1226 contained in the data plane mirror app tier 1240 can include a virtual network interface controller (VNIC) 1242 (e.g., the VNIC of 1142) that can execute a compute instance 1244 (e.g., similar to the compute instance 1144 of FIG. 11). The compute instance 1244 can facilitate communication between the app subnet(s) 1226 of the data plane mirror app tier 1240 and the app subnet(s) 1226 that can be contained in a data plane app tier 1246 (e.g., the data plane app tier 1146 of FIG. 11) via the VNIC 1242 contained in the data plane mirror app tier 1240 and the VNIC 1242 contained in the data plane app tier 1246.

The Internet gateway 1234 contained in the control plane VCN 1216 can be communicatively coupled to a metadata management service 1252 (e.g., the metadata management service 1152 of FIG. 11) that can be communicatively coupled to public Internet 1254 (e.g., public Internet 1154 of FIG. 11). Public Internet 1254 can be communicatively coupled to the NAT gateway 1238 contained in the control plane VCN 1216. The service gateway 1236 contained in the control plane VCN 1216 can be communicatively coupled to cloud services 1256 (e.g., cloud services 1156 of FIG. 11).

In some examples, the data plane VCN 1218 can be contained in the customer tenancy 1221. In this case, the IaaS provider may provide the control plane VCN 1216 for each customer, and the IaaS provider may, for each customer, set up a unique compute instance 1244 that is contained in the service tenancy 1219. Each compute instance 1244 may allow communication between the control plane VCN 1216, contained in the service tenancy 1219, and the data plane VCN 1218 that is contained in the customer tenancy 1221. The compute instance 1244 may allow resources, that are provisioned in the control plane VCN 1216 that is contained in the service tenancy 1219, to be deployed or otherwise used in the data plane VCN 1218 that is contained in the customer tenancy 1221.

In other examples, the customer of the IaaS provider may have databases that live in the customer tenancy 1221. In this example, the control plane VCN 1216 can include the data plane mirror app tier 1240 that can include app subnet(s) 1226. The data plane mirror app tier 1240 can reside in the data plane VCN 1218, but the data plane mirror app tier 1240 may not live in the data plane VCN 1218. That is, the data plane mirror app tier 1240 may have access to the customer tenancy 1221, but the data plane mirror app tier 1240 may not exist in the data plane VCN 1218 or be owned or operated by the customer of the IaaS provider. The data plane mirror app tier 1240 may be configured to make calls to the data plane VCN 1218 but may not be configured to make calls to any entity contained in the control plane VCN 1216. The customer may desire to deploy or otherwise use resources in the data plane VCN 1218 that are provisioned in the control plane VCN 1216, and the data plane mirror app tier 1240 can facilitate the desired deployment, or other usage of resources, of the customer.

In some embodiments, the customer of the IaaS provider can apply filters to the data plane VCN 1218. In this embodiment, the customer can determine what the data plane VCN 1218 can access, and the customer may restrict access to public Internet 1254 from the data plane VCN 1218. The IaaS provider may not be able to apply filters or otherwise control access of the data plane VCN 1218 to any outside networks or databases. Applying filters and controls by the customer onto the data plane VCN 1218, contained in the customer tenancy 1221, can help isolate the data plane VCN 1218 from other customers and from public Internet 1254.

In some embodiments, cloud services 1256 can be called by the service gateway 1236 to access services that may not exist on public Internet 1254, on the control plane VCN 1216, or on the data plane VCN 1218. The connection between cloud services 1256 and the control plane VCN 1216 or the data plane VCN 1218 may not be live or continuous. Cloud services 1256 may exist on a different network owned or operated by the IaaS provider. Cloud services 1256 may be configured to receive calls from the service gateway 1236 and may be configured to not receive calls from public Internet 1254. Some cloud services 1256 may be isolated from other cloud services 1256, and the control plane VCN 1216 may be isolated from cloud services 1256 that may not be in the same region as the control plane VCN 1216. For example, the control plane VCN 1216 may be located in “Region 1,” and cloud service “Deployment 11,” may be located in Region 1 and in “Region 2.” If a call to Deployment 11 is made by the service gateway 1236 contained in the control plane VCN 1216 located in Region 1, the call may be transmitted to Deployment 11 in Region 1. In this example, the control plane VCN 1216, or Deployment 11 in Region 1, may not be communicatively coupled to, or otherwise in communication with, Deployment 11 in Region 2.

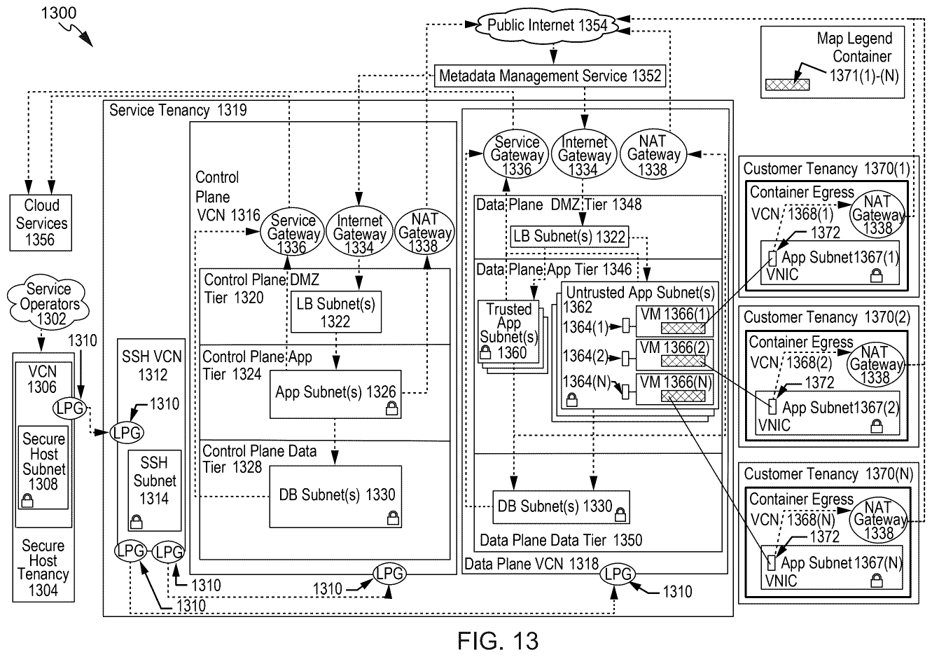

FIG. 13 is a block diagram 1300 illustrating another example pattern of an IaaS architecture, according to at least one embodiment. Service operators 1302 (e.g., service operators 1102 of FIG. 11) can be communicatively coupled to a secure host tenancy 1304 (e.g., the secure host tenancy 1104 of FIG. 11) that can include a virtual cloud network (VCN) 1306 (e.g., the VCN 1106 of FIG. 11) and a secure host subnet 1308 (e.g., the secure host subnet 1108 of FIG. 11). The VCN 1306 can include an LPG 1310 (e.g., the LPG 1110 of FIG. 11) that can be communicatively coupled to an SSH VCN 1312 (e.g., the SSH VCN 1112 of FIG. 11) via an LPG 1310 contained in the SSH VCN 1312. The SSH VCN 1312 can include an SSH subnet 1314 (e.g., the SSH subnet 1114 of FIG. 11), and the SSH VCN 1312 can be communicatively coupled to a control plane VCN 1316 (e.g., the control plane VCN 1116 of FIG. 11) via an LPG 1310 contained in the control plane VCN 1316 and to a data plane VCN 1318 (e.g., the data plane 1118 of FIG. 11) via an LPG 1310 contained in the data plane VCN 1318. The control plane VCN 1316 and the data plane VCN 1318 can be contained in a service tenancy 1319 (e.g., the service tenancy 1119 of FIG. 11).

The control plane VCN 1316 can include a control plane DMZ tier 1320 (e.g., the control plane DMZ tier 1120 of FIG. 11) that can include load balancer (LB) subnet(s) 1322 (e.g., LB subnet(s) 1122 of FIG. 11), a control plane app tier 1324 (e.g., the control plane app tier 1124 of FIG. 11) that can include app subnet(s) 1326 (e.g., similar to app subnet(s) 1126 of FIG. 11), a control plane data tier 1328 (e.g., the control plane data tier 1128 of FIG. 11) that can include DB subnet(s) 1330. The LB subnet(s) 1322 contained in the control plane DMZ tier 1320 can be communicatively coupled to the app subnet(s) 1326 contained in the control plane app tier 1324 and to an Internet gateway 1334 (e.g., the Internet gateway 1134 of FIG. 11) that can be contained in the control plane VCN 1316, and the app subnet(s) 1326 can be communicatively coupled to the DB subnet(s) 1330 contained in the control plane data tier 1328 and to a service gateway 1336 (e.g., the service gateway of FIG. 11) and a network address translation (NAT) gateway 1338 (e.g., the NAT gateway 1138 of FIG. 11). The control plane VCN 1316 can include the service gateway 1336 and the NAT gateway 1338.

The data plane VCN 1318 can include a data plane app tier 1346 (e.g., the data plane app tier 1146 of FIG. 11), a data plane DMZ tier 1348 (e.g., the data plane DMZ tier 1148 of FIG. 11), and a data plane data tier 1350 (e.g., the data plane data tier 1150 of FIG. 11). The data plane DMZ tier 1348 can include LB subnet(s) 1322 that can be communicatively coupled to trusted app subnet(s) 1360 and untrusted app subnet(s) 1362 of the data plane app tier 1346 and the Internet gateway 1334 contained in the data plane VCN 1318. The trusted app subnet(s) 1360 can be communicatively coupled to the service gateway 1336 contained in the data plane VCN 1318, the NAT gateway 1338 contained in the data plane VCN 1318, and DB subnet(s) 1330 contained in the data plane data tier 1350. The untrusted app subnet(s) 1362 can be communicatively coupled to the service gateway 1336 contained in the data plane VCN 1318 and DB subnet(s) 1330 contained in the data plane data tier 1350. The data plane data tier 1350 can include DB subnet(s) 1330 that can be communicatively coupled to the service gateway 1336 contained in the data plane VCN 1318.

The untrusted app subnet(s) 1362 can include one or more primary VNICs 1364(1)-(N) that can be communicatively coupled to tenant virtual machines (VMs) 1366(1)-(N). Each tenant VM 1366(1)-(N) can be communicatively coupled to a respective app subnet 1367(1)-(N) that can be contained in respective container egress VCNs 1368(1)-(N) that can be contained in respective customer tenancies 1370(1)-(N). Respective secondary VNICs 1372(1)-(N) can facilitate communication between the untrusted app subnet(s) 1362 contained in the data plane VCN 1318 and the app subnet contained in the container egress VCNs 1368(1)-(N). Each container egress VCNs 1368(1)-(N) can include a NAT gateway 1338 that can be communicatively coupled to public Internet 1354 (e.g., public Internet 1154 of FIG. 11).

The Internet gateway 1334 contained in the control plane VCN 1316 and contained in the data plane VCN 1318 can be communicatively coupled to a metadata management service 1352 (e.g., the metadata management system 1152 of FIG. 11) that can be communicatively coupled to public Internet 1354. Public Internet 1354 can be communicatively coupled to the NAT gateway 1338 contained in the control plane VCN 1316 and contained in the data plane VCN 1318. The service gateway 1336 contained in the control plane VCN 1316 and contained in the data plane VCN 1318 can be communicatively coupled to cloud services 1356.

In some embodiments, the data plane VCN 1318 can be integrated with customer tenancies 1370. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1346. Code to run the function may be executed in the VMs 1366(1)-(N), and the code may not be configured to run anywhere else on the data plane VCN 1318. Each VM 1366(1)-(N) may be connected to one customer tenancy 1370. Respective containers 1371(1)-(N) contained in the VMs 1366(1)-(N) may be configured to run the code. In this case, there can be a dual isolation (e.g., the containers 1371(1)-(N) running code, where the containers 1371(1)-(N) may be contained in at least the VM 1366(1)-(N) that are contained in the untrusted app subnet(s) 1362), which may help prevent incorrect or otherwise undesirable code from damaging the network of the IaaS provider or from damaging a network of a different customer. The containers 1371(1)-(N) may be communicatively coupled to the customer tenancy 1370 and may be configured to transmit or receive data from the customer tenancy 1370. The containers 1371(1)-(N) may not be configured to transmit or receive data from any other entity in the data plane VCN 1318. Upon completion of running the code, the IaaS provider may kill or otherwise dispose of the containers 1371(1)-(N).

In some embodiments, the trusted app subnet(s) 1360 may run code that may be owned or operated by the IaaS provider. In this embodiment, the trusted app subnet(s) 1360 may be communicatively coupled to the DB subnet(s) 1330 and be configured to execute CRUD operations in the DB subnet(s) 1330. The untrusted app subnet(s) 1362 may be communicatively coupled to the DB subnet(s) 1330, but in this embodiment, the untrusted app subnet(s) may be configured to execute read operations in the DB subnet(s) 1330. The containers 1371(1)-(N) that can be contained in the VM 1366(1)-(N) of each customer and that may run code from the customer may not be communicatively coupled with the DB subnet(s) 1330.

In other embodiments, the control plane VCN 1316 and the data plane VCN 1318 may not be directly communicatively coupled. In this embodiment, there may be no direct communication between the control plane VCN 1316 and the data plane VCN 1318. However, communication can occur indirectly through at least one method. An LPG 1310 may be established by the IaaS provider that can facilitate communication between the control plane VCN 1316 and the data plane VCN 1318. In another example, the control plane VCN 1316 or the data plane VCN 1318 can make a call to cloud services 1356 via the service gateway 1336. For example, a call to cloud services 1356 from the control plane VCN 1316 can include a request for a service that can communicate with the data plane VCN 1318.

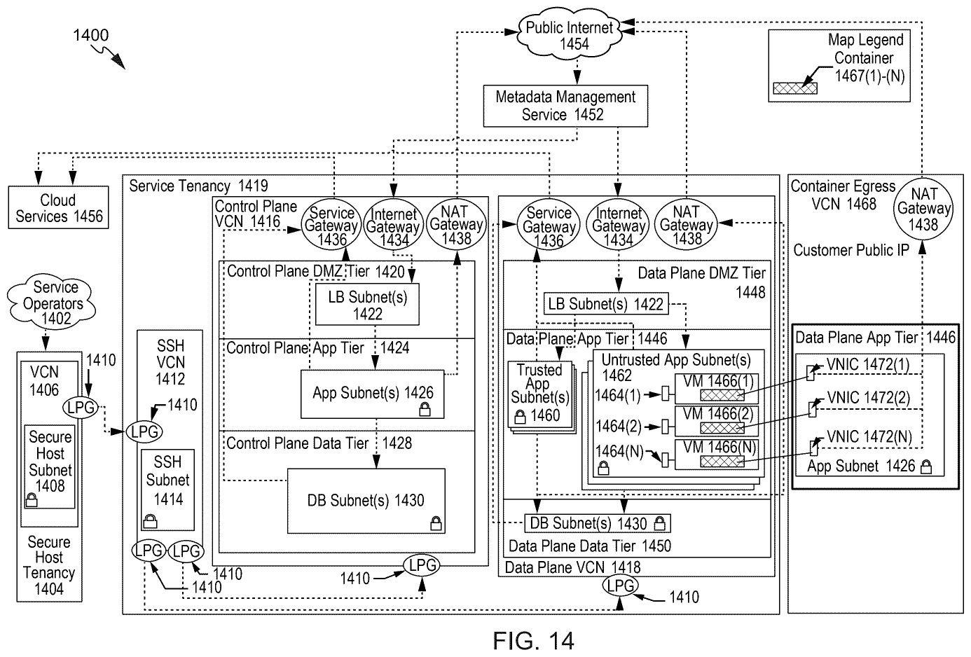

FIG. 14 is a block diagram 1400 illustrating another example pattern of an IaaS architecture, according to at least one embodiment. Service operators 1402 (e.g., service operators 1102 of FIG. 11) can be communicatively coupled to a secure host tenancy 1404 (e.g., the secure host tenancy 1104 of FIG. 11) that can include a virtual cloud network (VCN) 1406 (e.g., the VCN 1106 of FIG. 11) and a secure host subnet 1408 (e.g., the secure host subnet 1108 of FIG. 11). The VCN 1406 can include an LPG 1410 (e.g., the LPG 1110 of FIG. 11) that can be communicatively coupled to an SSH VCN 1412 (e.g., the SSH VCN 1112 of FIG. 11) via an LPG 1410 contained in the SSH VCN 1412. The SSH VCN 1412 can include an SSH subnet 1414 (e.g., the SSH subnet 1114 of FIG. 11), and the SSH VCN 1412 can be communicatively coupled to a control plane VCN 1416 (e.g., the control plane VCN 1116 of FIG. 11) via an LPG 1410 contained in the control plane VCN 1416 and to a data plane VCN 1418 (e.g., the data plane 1118 of FIG. 11) via an LPG 1410 contained in the data plane VCN 1418. The control plane VCN 1416 and the data plane VCN 1418 can be contained in a service tenancy 1419 (e.g., the service tenancy 1119 of FIG. 11).

The control plane VCN 1416 can include a control plane DMZ tier 1420 (e.g., the control plane DMZ tier 1120 of FIG. 11) that can include LB subnet(s) 1422 (e.g., LB subnet(s) 1122 of FIG. 11), a control plane app tier 1424 (e.g., the control plane app tier 1124 of FIG. 11) that can include app subnet(s) 1426 (e.g., app subnet(s) 1126 of FIG. 11), a control plane data tier 1428 (e.g., the control plane data tier 1128 of FIG. 11) that can include DB subnet(s) 1430 (e.g., DB subnet(s) 1330 of FIG. 13). The LB subnet(s) 1422 contained in the control plane DMZ tier 1420 can be communicatively coupled to the app subnet(s) 1426 contained in the control plane app tier 1424 and to an Internet gateway 1434 (e.g., the Internet gateway 1134 of FIG. 11) that can be contained in the control plane VCN 1416, and the app subnet(s) 1426 can be communicatively coupled to the DB subnet(s) 1430 contained in the control plane data tier 1428 and to a service gateway 1436 (e.g., the service gateway of FIG. 11) and a network address translation (NAT) gateway 1438 (e.g., the NAT gateway 1138 of FIG. 11). The control plane VCN 1416 can include the service gateway 1436 and the NAT gateway 1438.

The data plane VCN 1418 can include a data plane app tier 1446 (e.g., the data plane app tier 1146 of FIG. 11), a data plane DMZ tier 1448 (e.g., the data plane DMZ tier 1148 of FIG. 11), and a data plane data tier 1450 (e.g., the data plane data tier 1150 of FIG. 11). The data plane DMZ tier 1448 can include LB subnet(s) 1422 that can be communicatively coupled to trusted app subnet(s) 1460 (e.g., trusted app subnet(s) 1360 of FIG. 13) and untrusted app subnet(s) 1462 (e.g., untrusted app subnet(s) 1362 of FIG. 13) of the data plane app tier 1446 and the Internet gateway 1434 contained in the data plane VCN 1418. The trusted app subnet(s) 1460 can be communicatively coupled to the service gateway 1436 contained in the data plane VCN 1418, the NAT gateway 1438 contained in the data plane VCN 1418, and DB subnet(s) 1430 contained in the data plane data tier 1450. The untrusted app subnet(s) 1462 can be communicatively coupled to the service gateway 1436 contained in the data plane VCN 1418 and DB subnet(s) 1430 contained in the data plane data tier 1450. The data plane data tier 1450 can include DB subnet(s) 1430 that can be communicatively coupled to the service gateway 1436 contained in the data plane VCN 1418.

The untrusted app subnet(s) 1462 can include primary VNICs 1464(1)-(N) that can be communicatively coupled to tenant virtual machines (VMs) 1466(1)-(N) residing within the untrusted app subnet(s) 1462. Each tenant VM 1466(1)-(N) can run code in a respective container 1467(1)-(N), and be communicatively coupled to an app subnet 1426 that can be contained in a data plane app tier 1446 that can be contained in a container egress VCN 1468. Respective secondary VNICs 1472(1)-(N) can facilitate communication between the untrusted app subnet(s) 1462 contained in the data plane VCN 1418 and the app subnet contained in the container egress VCN 1468. The container egress VCN can include a NAT gateway 1438 that can be communicatively coupled to public Internet 1454 (e.g., public Internet 1154 of FIG. 11).

The Internet gateway 1434 contained in the control plane VCN 1416 and contained in the data plane VCN 1418 can be communicatively coupled to a metadata management service 1452 (e.g., the metadata management system 1152 of FIG. 11) that can be communicatively coupled to public Internet 1454. Public Internet 1454 can be communicatively coupled to the NAT gateway 1438 contained in the control plane VCN 1416 and contained in the data plane VCN 1418. The service gateway 1436 contained in the control plane VCN 1416 and contained in the data plane VCN 1418 can be communicatively coupled to cloud services 1456.

In some examples, the pattern illustrated by the architecture of block diagram 1400 of FIG. 14 may be considered an exception to the pattern illustrated by the architecture of block diagram 1300 of FIG. 13 and may be desirable for a customer of the IaaS provider if the IaaS provider cannot directly communicate with the customer (e.g., a disconnected region). The respective containers 1467(1)-(N) that are contained in the VMs 1466(1)-(N) for each customer can be accessed in real-time by the customer. The containers 1467(1)-(N) may be configured to make calls to respective secondary VNICs 1472(1)-(N) contained in app subnet(s) 1426 of the data plane app tier 1446 that can be contained in the container egress VCN 1468. The secondary VNICs 1472(1)-(N) can transmit the calls to the NAT gateway 1438 that may transmit the calls to public Internet 1454. In this example, the containers 1467(1)-(N) that can be accessed in real-time by the customer can be isolated from the control plane VCN 1416 and can be isolated from other entities contained in the data plane VCN 1418. The containers 1467(1)-(N) may also be isolated from resources from other customers.

In other examples, the customer can use the containers 1467(1)-(N) to call cloud services 1456. In this example, the customer may run code in the containers 1467(1)-(N) that requests a service from cloud services 1456. The containers 1467(1)-(N) can transmit this request to the secondary VNICs 1472(1)-(N) that can transmit the request to the NAT gateway that can transmit the request to public Internet 1454. Public Internet 1454 can transmit the request to LB subnet(s) 1422 contained in the control plane VCN 1416 via the Internet gateway 1434. In response to determining the request is valid, the LB subnet(s) can transmit the request to app subnet(s) 1426 that can transmit the request to cloud services 1456 via the service gateway 1436.

It should be appreciated that IaaS architectures 1100, 1200, 1300, 1400 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

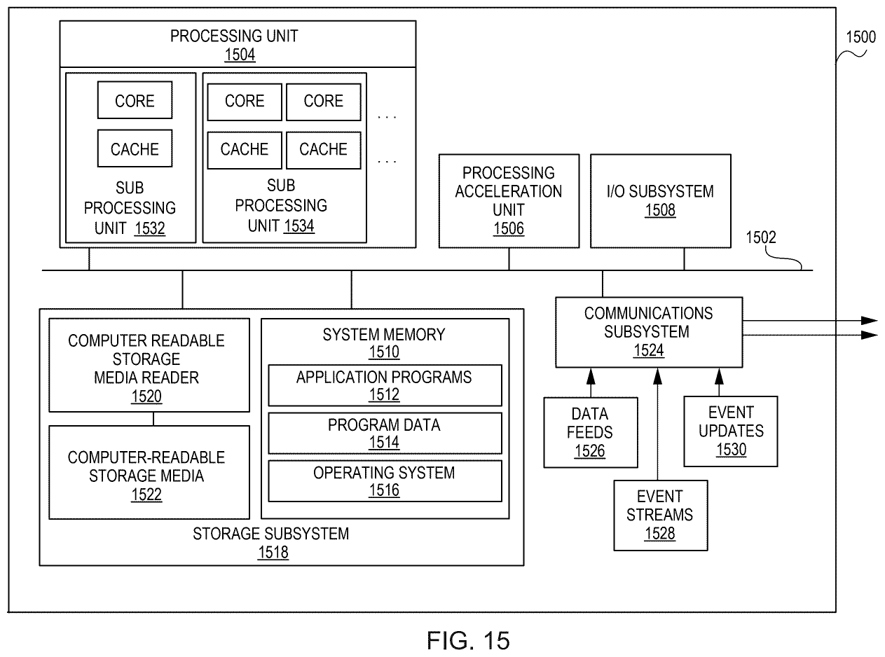

FIG. 15 illustrates an example computer system 1500, in which various embodiments may be implemented. The system 1500 may be used to implement any of the computer systems described above. As shown in the figure, computer system 1500 includes a processing unit 1504 that communicates with a number of peripheral subsystems via a bus subsystem 1502. These peripheral subsystems may include a processing acceleration unit 1506, an I/O subsystem 1508, a storage subsystem 1518 and a communications subsystem 1524. Storage subsystem 1518 includes tangible computer-readable storage media 1522 and a system memory 1510.

Bus subsystem 1502 provides a mechanism for letting the various components and subsystems of computer system 1500 communicate with each other as intended. Although bus subsystem 1502 is shown schematically as a single bus, alternative embodiments of the bus subsystem may utilize multiple buses. Bus subsystem 1502 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1504, which can be implemented as one or more integrated circuits (e.g., a conventional microprocessor or microcontroller), controls the operation of computer system 1500. One or more processors may be included in processing unit 1504. These processors may include single core or multicore processors. In certain embodiments, processing unit 1504 may be implemented as one or more independent processing units 1532 and/or 1534 with single or multicore processors included in each processing unit. In other embodiments, processing unit 1504 may also be implemented as a quad-core processing unit formed by integrating two dual-core processors into a single chip.

In various embodiments, processing unit 1504 can execute a variety of programs in response to program code and can maintain multiple concurrently executing programs or processes. At any given time, some or all of the program code to be executed can be resident in processor(s) 1504 and/or in storage subsystem 1518. Through suitable programming, processor(s) 1504 can provide various functionalities described above. Computer system 1500 may additionally include a processing acceleration unit 1506, which can include a digital signal processor (DSP), a special-purpose processor, and/or the like.

I/O subsystem 1508 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1500 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1500 may comprise a storage subsystem 1518 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1504 provide the functionality described above. Storage subsystem 1518 may also provide a repository for storing data used in accordance with the present disclosure.

As depicted in the example in FIG. 15, storage subsystem 1518 can include various components including a system memory 1510, computer-readable storage media 1522, and a computer readable storage media reader 1520. System memory 1510 may store program instructions that are loadable and executable by processing unit 1504. System memory 1510 may also store data that is used during the execution of the instructions and/or data that is generated during the execution of the program instructions. Various different kinds of programs may be loaded into system memory 1510 including but not limited to client applications, Web browsers, mid-tier applications, relational database management systems (RDBMS), virtual machines, containers, etc.

System memory 1510 may also store an operating system 1516. Examples of operating system 1516 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1500 executes one or more virtual machines, the virtual machines along with their guest operating systems (GOSs) may be loaded into system memory 1510 and executed by one or more processors or cores of processing unit 1504.

System memory 1510 can come in different configurations depending upon the type of computer system 1500. For example, system memory 1510 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1510 may include a basic input/output system (BIOS) containing basic routines that help to transfer information between elements within computer system 1500, such as during start-up.

Computer-readable storage media 1522 may represent remote, local, fixed, and/or removable storage devices plus storage media for temporarily and/or more permanently containing, storing, computer-readable information for use by computer system 1500 including instructions executable by processing unit 1504 of computer system 1500.

Computer-readable storage media 1522 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1522 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1522 may include, but is not limited to, Zip® drives, flash memory cards, universal serial bus (USB) flash drives, secure digital (SD) cards, DVD disks, digital video tape, and the like. Computer-readable storage media 1522 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1500.

Machine-readable instructions executable by one or more processors or cores of processing unit 1504 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1524 provides an interface to other computer systems and networks. Communications subsystem 1524 serves as an interface for receiving data from and transmitting data to other systems from computer system 1500. For example, communications subsystem 1524 may enable computer system 1500 to connect to one or more devices via the Internet. In some embodiments communications subsystem 1524 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1524 can provide wired network connectivity (e.g., Ethernet) in addition to or instead of a wireless interface.

In some embodiments, communications subsystem 1524 may also receive input communication in the form of structured and/or unstructured data feeds 1526, event streams 1528, event updates 1530, and the like on behalf of one or more users who may use computer system 1500.

By way of example, communications subsystem 1524 may be configured to receive data feeds 1526 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1524 may also be configured to receive data in the form of continuous data streams, which may include event streams 1528 of real-time events and/or event updates 1530, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1524 may also be configured to output the structured and/or unstructured data feeds 1526, event streams 1528, event updates 1530, and the like to one or more databases that may be in communication with one or more streaming data source computers coupled to computer system 1500.

Computer system 1500 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1500 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed:

1. A computer-implemented method comprising:
accessing medical text associated with a patient;
annotating spans in the medical text for drug and event entities to create labelled medical text;
linking, using entity linking, the spans in the labelled medical text to concepts in an ontology, wherein the linking comprises:
for a subject span in the labelled medical text with a mapped entity type of drug or event, link the subject span to a most similar subject concept in the ontology, and
for an object span in the labelled medical text with a mapped entity type of drug or event, link the object span to a most similar object concept in the ontology;
for each pair of linked subject and object spans, identifying possible paths in the ontology between the subject concept and the object concept, ranking each of the possible paths in the ontology, identifying a top number of paths from all the possible paths based on the ranking, and concatenating a verbalized form of each path of the top number of paths with the subject span and the object span from the labelled medical text to create an ontology augmented text instance corresponding to the pair of linked subject and object spans;
generating a prompt comprising the medical text and each ontology augmented text instance;
generating, by a generative artificial intelligence model, one or more adverse drug reaction relation predictions for the patient based on the prompt; and
providing the one or more adverse drug reaction relation predictions to a user.

2. The computer-implemented method of claim 1, wherein the ontology comprises concepts, relations, and one or more triplets having a subject concept, a relation, and an object concept, and wherein a path in the ontology is a sequence of concepts and relations identified therein by associated one or more triplets.

3. The computer-implemented method of claim 2, further comprising:
retrieving, based on a query of ontology data, an ontology associated with the medical text;
verbalizing, using one or more templates, the one or more triplets in the ontology to represent each of the one or more triplets in form of a textual description;
embedding the textual description for each of the one or more triplets; and
storing the embedded textual description for each of the one or more triplets in a vector database, wherein the linking is performed between the spans in the labelled medical text to the concepts in the embedded textual description for each of the one or more triplets stored in the vector database.

4. The computer-implemented method of claim 3, wherein a determination of whether a subject span or an object span in the labelled medical text with the mapped entity type of drug or event is most similar to a corresponding subject concept or object concept in the ontology is performed using an approximate nearest neighbor (ANN) search, and wherein the ANN search performs a semantic similarity search in the vector database using the subject spans and the object spans as query data points.

5. The computer-implemented method of claim 4, wherein ranking each of the possible paths in the ontology comprises:
verbalizing each of the identified possible paths in the ontology by concatenating the textual description for the one or more triplets associated with each of the identified possible paths;
concatenating each pair of the linked subject and object spans with the associated verbalized form of each of the identified possible paths to generate a corresponding possible path input;
determining, by a pre-trained cross-encoder, a scaler value for each possible path input; and
ranking the possible path inputs based on the corresponding scalar value for each possible path input, wherein the top number of paths are identified from all the possible paths based on the ranking of the possible path inputs.

6. The computer-implemented method of claim 1, wherein the prompt further comprises an overview of the adverse drug reaction relation task and intermediate texts as part of in-context learning, and wherein the generative artificial intelligence model is a LLM fine-tuned for the adverse drug reaction relation task.

7. The computer-implemented method of claim 1, wherein the one or more adverse drug reaction relation predictions are provided to a user as part of plug-in for an application associated with a cloud service.

8. A system comprising:
one or more processors;
a memory coupled to the one or more processors, the memory storing a plurality of instructions executable by the one or more processors, the plurality of instructions comprising instructions that when executed by the one or more processors cause the one or more processors to perform operations comprising:
accessing medical text associated with a patient;
annotating spans in the medical text for drug and event entities to create labelled medical text;
linking, using entity linking, the spans in the labelled medical text to concepts in an ontology, wherein the linking comprises:
for a subject span in the labelled medical text with a mapped entity type of drug or event, link the subject span to a most similar subject concept in the ontology, and
for an object span in the labelled medical text with a mapped entity type of drug or event, link the object span to a most similar object concept in the ontology;
for each pair of linked subject and object spans, identifying possible paths in the ontology between the subject concept and the object concept, ranking each of the possible paths in the ontology, identifying a top number of paths from all the possible paths based on the ranking, and concatenating a verbalized form of each path of the top number of paths with the subject span and the object span from the labelled medical text to create an ontology augmented text instance corresponding to the pair of linked subject and object spans;
generating a prompt comprising the medical text and each ontology augmented text instance;
generating, by a generative artificial intelligence model, one or more adverse drug reaction relation predictions for the patient based on the prompt; and
providing the one or more adverse drug reaction relation predictions to a user.

9. The system of claim 8, wherein the ontology comprises concepts, relations, and one or more triplets having a subject concept, a relation, and an object concept, and wherein a path in the ontology is a sequence of concepts and relations identified therein by associated one or more triplets.

10. The system of claim 9, wherein the operations further comprise:
retrieving, based on a query of ontology data, an ontology associated with the medical text;
verbalizing, using one or more templates, the one or more triplets in the ontology to represent each of the one or more triplets in form of a textual description;
embedding the textual description for each of the one or more triplets; and
storing the embedded textual description for each of the one or more triplets in a vector database, wherein the linking is performed between the spans in the labelled medical text to the concepts in the embedded textual description for each of the one or more triplets stored in the vector database.

11. The system of claim 10, wherein a determination of whether a subject span or an object span in the labelled medical text with the mapped entity type of drug or event is most similar to a corresponding subject concept or object concept in the ontology is performed using an approximate nearest neighbor (ANN) search, and wherein the ANN search performs a semantic similarity search in the vector database using the subject spans and the object spans as query data points.

12. The system of claim 11, wherein ranking each of the possible paths in the ontology comprises:
verbalizing each of the identified possible paths in the ontology by concatenating the textual description for the one or more triplets associated with each of the identified possible paths;
concatenating each pair of the linked subject and object spans with the associated verbalized form of each of the identified possible paths to generate a corresponding possible path input;
determining, by a pre-trained cross-encoder, a scaler value for each possible path input; and
ranking the possible path inputs based on the corresponding scalar value for each possible path input, wherein the top number of paths are identified from all the possible paths based on the ranking of the possible path inputs.

13. The system of claim 8, wherein the prompt further comprises an overview of the adverse drug reaction relation task and intermediate texts as part of in-context learning, and wherein the generative artificial intelligence model is a LLM fine-tuned for the adverse drug reaction relation task.

14. The system of claim 8, wherein the one or more adverse drug reaction relation predictions are provided to a user as part of plug-in for an application associated with a cloud service.

15. A computer-program product tangibly embodied in a non-transitory machine-readable storage medium, including instructions configured to cause one or more data processors to perform operations comprising:
accessing medical text associated with a patient;
annotating spans in the medical text for drug and event entities to create labelled medical text;
linking, using entity linking, the spans in the labelled medical text to concepts in an ontology, wherein the linking comprises:
for a subject span in the labelled medical text with a mapped entity type of drug or event, link the subject span to a most similar subject concept in the ontology, and
for an object span in the labelled medical text with a mapped entity type of drug or event, link the object span to a most similar object concept in the ontology;
for each pair of linked subject and object spans, identifying possible paths in the ontology between the subject concept and the object concept, ranking each of the possible paths in the ontology, identifying a top number of paths from all the possible paths based on the ranking, and concatenating a verbalized form of each path of the top number of paths with the subject span and the object span from the labelled medical text to create an ontology augmented text instance corresponding to the pair of linked subject and object spans;
generating a prompt comprising the medical text and each ontology augmented text instance;
generating, by a generative artificial intelligence model, one or more adverse drug reaction relation predictions for the patient based on the prompt; and
providing the one or more adverse drug reaction relation predictions to a user.

16. The computer-implemented method of claim 15, wherein the ontology comprises concepts, relations, and one or more triplets having a subject concept, a relation, and an object concept, and wherein a path in the ontology is a sequence of concepts and relations identified therein by associated one or more triplets.

17. The computer-program product of claim 16, wherein the operations further comprise:
retrieving, based on a query of ontology data, an ontology associated with the medical text;
verbalizing, using one or more templates, the one or more triplets in the ontology to represent each of the one or more triplets in form of a textual description;
embedding the textual description for each of the one or more triplets; and
storing the embedded textual description for each of the one or more triplets in a vector database, wherein the linking is performed between the spans in the labelled medical text to the concepts in the embedded textual description for each of the one or more triplets stored in the vector database.

18. The computer-program product of claim 17, wherein a determination of whether a subject span or an object span in the labelled medical text with the mapped entity type of drug or event is most similar to a corresponding subject concept or object concept in the ontology is performed using an approximate nearest neighbor (ANN) search, and wherein the ANN search performs a semantic similarity search in the vector database using the subject spans and the object spans as query data points.

19. The computer-program product of claim 18, wherein ranking each of the possible paths in the ontology comprises:
verbalizing each of the identified possible paths in the ontology by concatenating the textual description for the one or more triplets associated with each of the identified possible paths;
concatenating each pair of the linked subject and object spans with the associated verbalized form of each of the identified possible paths to generate a corresponding possible path input;
determining, by a pre-trained cross-encoder, a scaler value for each possible path input; and
ranking the possible path inputs based on the corresponding scalar value for each possible path input, wherein the top number of paths are identified from all the possible paths based on the ranking of the possible path inputs.

20. The computer-program product of claim 15, wherein the prompt further comprises an overview of the adverse drug reaction relation task and intermediate texts as part of in-context learning, and wherein the generative artificial intelligence model is a LLM fine-tuned for the adverse drug reaction relation task.
